(19) United States
(12) Patent Application Publication
(10) Pub. No.: US 2024/0420326 A1
(43) Pub. Date: Dec. 19, 2024

(54) SYSTEMS, METHODS AND COMPUTER-ACCESSIBLE MEDIUM FOR DETERMINING AND/OR ANALYZING CANCER OUTCOME(S) AND/OR TREATMENT RESPONSES

(71) Applicant: NEW YORK UNIVERSITY, New York, NY (US)

(72) Inventors: JAN WITOWSKI, New York, NY (US); KRZYSZTOF J. GERAS, Hoboken, NJ (US)

(21) Appl. No.: 18/743,715

(22) Filed: Jun. 14, 2024

Related U.S. Application Data
(60) Provisional application No. 63/521,262, filed on Jun. 15, 2023.

(51) Int. Cl. G06T 7/00; G16H 50/20

(52) U.S. Cl. CPC G06T 7/0012; G16H 50/20; G06T 2207/10088; G06T 2207/20081; G06T 2207/30024; G06T 2207/30068

(57) ABSTRACT

Exemplary methods, systems, procedures, computer programs, kits, devices, and computer-executable code according to exemplary embodiments of the present disclosure can be provided for predicting cancer outcomes and treatment response. Thus, exemplary systems, methods, and computer-accessible medium can be provided that can extract at least one feature from a medical image of a tissue to provide an extracted image feature, extract at least one feature from digitized histopathology data of the tissue to provide an extracted histopathology feature, process the extracted image feature and the extracted histopathology feature to provide a multi-modal representation of the tissue, and process the multimodal representation of the tissue with a specific procedure to provide a prediction regarding a medical outcome of the tissue.

### CROSS REFERENCE TO RELATED APPLICATION(S)

This application relates to and claims the benefit of priority from U.S. Provisional Patent Application No. 63/521,262, filed on Jun. 15, 2023, the entire disclosure of which is incorporated herein by reference in its entirety

### FIELD OF THE DISCLOSURE

The present disclosure relates to systems, methods and computer-accessible medium for analyzing information associated with cancer, and more particularly to systems, methods and computer-accessible medium for predicting cancer outcome(s) and/or treatment response(s).

### BACKGROUND INFORMATION

Breast magnetic resonance imaging (MRI) is a highly sensitive modality for detecting breast cancer with a reported sensitivity of more than 80%. Use in screening is often limited to high-risk patients. Diagnostic MRI is also useful for additional indications such as problem solving and patients with recently diagnosed breast cancer.

Thus, it may be beneficial to provide exemplary systems, methods and computer-accessible mediums, which can determine and/or learn to predict risk of cancer recurrence, diagnose cancer or cancer subtype, and/or predict treatment response(s).

### SUMMARY OF EXEMPLARY EMBODIMENTS

According some exemplary embodiments of the present disclosure, a system can be provided which can comprise, e.g., (a) a first feature extractor that extracts at least one feature from a medical image of a tissue to provide an extracted image feature, (b) a second feature extractor that extracts at least one feature from digitized histopathology data of the tissue to provide an extracted histopathology feature. and (c) a processing unit. For example, the processing unit can: (1) processes the extracted image feature and the extracted histopathology feature to provide a multi-modal representation of the tissue, and (2) processes the multimodal representation of the tissue with a procedure to provide a prediction regarding a medical outcome of the tissue.

In additional exemplary embodiments of the present disclosure, a method and/or a computer program product (comprising a non-transitory, computer-readable medium having a computer-readable program encoded therein, the computer readable program adapted to be executed to implement a method) which can e.g., utilize a medical image analysis system, which can comprises: (1) an image receiving portal, (2) a first feature extractor, (3) a histopathology data receiving portal, (4) a second feature extractor, and (5) an output function.

For example, the method and the computer program product can be used to receive—by the image receiving portal—a medical image of a tissue from an image input source. It is also possible to extract—by the first feature extractor—at least one feature from the medical image of the tissue to provide an extracted image feature. The histopathology data receiving portal can be used to receive digitized histopathology data of the tissue from a histopathology data source. The second feature extractor can be used to extract at least one feature from the digitized histopathology data of the tissue to provide an extracted histopathology feature. The extracted image feature and the extracted histopathology feature can be processed to provide a multi-modal representation of the tissue. The multimodal representation of the tissue can be processed with a particular procedure to provide a prediction regarding a medical outcome of the tissue. Further, the output function can be used to output the prediction regarding the medical outcome of the tissue.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an exemplary diagram of a computer-implemented system for generating predictions about breast cancer outcomes according to an exemplary embodiment of the present disclosure;

FIG. 2 is an exemplary diagram of supervised pre-training according to an exemplary embodiment of the present disclosure;

FIG. 3 is an exemplary diagram of self-supervised pretraining according to an exemplary embodiment of the present disclosure;

FIG. 4 is an exemplary diagram of Downstream Model training according to an exemplary embodiment of the present disclosure;

FIG. 5 is an exemplary diagram of treatment response prediction according to an exemplary embodiment of the present disclosure;

FIG. 6 is an exemplary diagram of a Web Application interface workflow according to an exemplary embodiment of the present disclosure;

FIG. 7 is an exemplary diagram of an exemplary integration between the Web Application, hospital systems, and artificial intelligence (AI) system according to an exemplary embodiment of the present disclosure;

FIG. 8 is an illustration of an exemplary use of the feature extractor to predict the probability of breast cancer in DCE-MRI examinations according to an exemplary embodiment of the present disclosure;

FIG. 9 is a set of exemplary graphs providing the feature extractor's exemplary performance on internal and external test sets according to an exemplary embodiment of the present disclosure;

FIG. 10 is an exemplary illustration of the feature extractor's exemplary performance in key subgroups on the internal test set according to an exemplary embodiment of the present disclosure;

FIG. 11 is a set of graphs providing exemplary results of the DCA support using the feature extractor for making diagnostic decisions in low-risk patients with BI-RADS 4 lesions according to an exemplary embodiment of the present disclosure;

FIG. 12 is a set of graphs providing exemplary receiver operating characteristic (ROC) and precision-recall (PR) curves from the reader study on the N subset according to an exemplary embodiment of the present disclosure;

FIGS. 13 and 14 are exemplary graphs illustrating that hybrid predictions are stronger than readers' predictions alone, according to exemplary embodiments of the present disclosure.

FIGS. 15 and 16 are exemplary graphs illustrating an exemplary performance of a hybrid mode as a function of α∈(0, 99]% according to exemplary embodiments of the present disclosure;

FIG. 17 is a graph illustrating exemplary changes in Fleiss' kappa for hybrids with a fixed scalar, depending on the scalar value according to an exemplary embodiment of the present disclosure;

FIGS. 18-20 are exemplary graphs illustrating exemplary ROC curves for all readers and hybrids according to exemplary embodiments of the present disclosure;

FIG. 21 is an exemplary graph illustrating exemplary empirical ROC curves for subgroups per background parenchymal enhancement category according to exemplary embodiments of the present disclosure;

FIG. 22 is an exemplary graph illustrating exemplary empirical ROC curves for subgroups for an exam indication according to exemplary embodiments of the present disclosure;

FIG. 23 is an exemplary graph illustrating exemplary empirical ROC curves for subgroups for a cancer histological subtype according to exemplary embodiments of the present disclosure;

FIG. 24 is an exemplary graph illustrating exemplary empirical ROC curves for subgroups for a cancer molecular subtype according to exemplary embodiments of the present disclosure;

FIG. 25 is an exemplary graph illustrating exemplary empirical ROC curves for subgroups for BI-RADS category according to exemplary embodiments of the present disclosure;

FIG. 26 is an exemplary graph illustrating exemplary empirical ROC curves for subgroups for race according to exemplary embodiments of the present disclosure;

FIG. 27 is an exemplary graph illustrating empirical precision-recall curves for subgroups per background parenchymal enhancement category according to exemplary embodiments of the present disclosure;

FIG. 28 is an exemplary graph illustrating empirical precision-recall curves for an exam indication according to exemplary embodiments of the present disclosure;

FIG. 29 is an exemplary graph illustrating empirical precision-recall curves for a cancer histological subtype according to exemplary embodiments of the present disclosure;

FIG. 30 is an exemplary graph illustrating empirical precision-recall curves for a cancer molecular subtype according to exemplary embodiments of the present disclosure;

FIG. 31 is an exemplary graph illustrating empirical precision-recall curves for a BIRADS category according to exemplary embodiments of the present disclosure;

FIG. 32 is an exemplary graph illustrating empirical precision-recall curves for a race according to exemplary embodiments of the present disclosure;

FIGS. 33 and 34 exemplary graphs which illustrate trade-off in missed cancers versus correctly avoided biopsies when using only the feature extractor to decide on management according to exemplary embodiments of the present disclosure;

FIG. 35 is an illustration of an exemplary case study according to an exemplary embodiment of the present disclosure;

FIG. 36 is an illustration of an exemplary case study according to an exemplary embodiment of the present disclosure;

FIG. 37 is an illustration of an exemplary case study according to an exemplary embodiment of the present disclosure;

FIG. 38 is a set of illustrations of two exemplary case studies according to exemplary embodiments of the present disclosure;

FIG. 39 is an illustration of an exemplary case study according to an exemplary embodiment of the present disclosure; and

FIG. 40 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the Figures, it is done so in connection with the illustrative embodiments and is not limited by the particular embodiments illustrated in the Figures and the appended claims.

### DETAILED DESCRIPTION OF EXEMPLARY EMBODIMENTS

The following description of exemplary embodiments provides non-limiting representative examples referencing numerals to particularly describe features and teachings of different aspects of the present disclosure. The exemplary embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the exemplary embodiments of the present disclosure to such an extent that other implementations, not specifically covered but within the knowledge of a person of skill in the art having read the description of embodiments, would be understood to be consistent with an application of the exemplary embodiments of the present disclosure.

Exemplary methods, systems, methods, computer programs, kits, devices, and computer-executable code for predicting cancer outcomes and treatment response according to the exemplary embodiments of the present disclosure are described herein. In some exemplary embodiments, the exemplary cancer outcomes can comprise cancer diagnosis, cancer staging, cancer recurrence, response to treatment, treatment benefit, and prognosis.

### Exemplary Method of Cancer Prediction

According to certain exemplary embodiments of the present disclosure, systems, methods and computer-accessible medium can be provided for diagnosing and/or predicting a condition or disease in a subject. Exemplary systems, methods and computer-accessible medium can also be provided for predicting treatment response of a condition and/or a disease in a subject. Further exemplary systems, methods and computer-accessible medium according to the present disclosure can be provided for predicting recurrence of a condition and/or a disease in a subject.

In some exemplary embodiments of the present disclosure, the condition or disease can be cancer. In For example, the cancer can be a solid tumor, a hematological cancer, a metastatic cancer, a soft tissue tumor, or a combination thereof. Additionally, or alternatively, the cancer can be the solid tumor, and wherein the solid tumor is selected from the group consisting of melanoma, pancreatic cancer, breast cancer, colorectal cancer, lung cancer, skin cancer, ovarian cancer, liver cancer, and a combination thereof. Further or in addition, the cancer can be the hematological cancer, and the hematological cancer can be selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and a combination thereof. In some exemplary embodiments of the present disclosure, the Non-Hodgkin's lymphoma can be selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma, Burkitt lymphoma, lymphoplasmacytic lymphoma, hairy cell leukemia, and a combination thereof. For example, the T-cell lymphoma can be peripheral T-cell lymphoma.

According to further exemplary embodiments of the present disclosure, the cancer is breast cancer. In some exemplary embodiments of the present disclosure, the breast cancer can be a subtype of breast cancer. Additionally, or alternatively, the breast cancer can be a luminal breast cancer. Luminal breast cancers can include luminal A, luminal B, or luminal C subtypes. In some exemplary embodiments of the present disclosure, the cancer is a nonluminal breast cancer. Nonluminal breast cancer can include triple negative breast cancer, HER2 enriched breast cancer, or nonluminal unknown breast cancer. The subtype of the breast cancer may be unknown.

In some exemplary embodiments of the present disclosure, the breast cancer can be classified based on histological type. Breast cancer histological types can include ductal carcinoma in situ, invasive ductal carcinoma, metastatic carcinoma, adenocarcinoma, invasive lobular carcinoma, invasive mammary carcinoma, papillary carcinoma, or lymphoma. For example, the histological type of the breast cancer can be unknown.

Non-limiting examples of cancers that can be predicted with exemplary embodiments of the systems, methods and computer-accessible medium according to the present disclosure can include cancer cells from the bladder, blood, bone, bone marrow, brain, breast, colon, esophagus, gastrointestine, gum, head, kidney, liver, lung, nasopharynx, neck, ovary, prostate, skin, stomach, pancreas, prostate testis, tongue, cervix, or uterus. Non-limiting examples of cancer histological types can include neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some exemplary embodiments, the tumor may comprise an osteosarcoma, angiosarcoma, rhabdosarcoma, leiomyosarcoma, Ewing sarcoma, glioblastoma, neuroblastoma, or leukemia.

In some exemplary embodiments of the present disclosure, the cancer can be characterized by a cancer antigen present on the cancer. For example, the cancer antigen can be a tumor antigen, a stromal antigen, or a hematological antigen. In some exemplary embodiments, the cancer antigen is selected from the group consisting of BCMA, CD19, CD20, CD22, CD30, CD33, FcRH5, PDL1, CD47, CD117 (c-kit), gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, calcitonin, Cyclin-B1, Cyclin D1, DCP, 9D7, Ep-CAM, EphA3, Gastrin, HE4, Her2/neu, Telomerase, SAP-1, Survivin, NY-ESO-1/LAGE-1, PRAME, SSX-2, Melan-A/MART-1, 5-HIAA, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA15-3, CA27.29, CA19-9, CA-125, BAGE, GAGE, NY-ESO-1, β-catenin, CDK4, CDC27, a actinin-4, TRP1/gp75, TRP2, gp100, lactate dehydrogenase, Melan-A/MARTI, gangliosides, WT1, EphA3, Epidermal growth factor receptor (EGFR), MART-2, MART-1, MPO, MUC1, MUC2, MUM1, MUM2, MUM3, NA88-1, nuclear matrix protein 22, NPM, OA1, OGT, PAP, PD-L1, RCC, RUI1, RUI2, SAGE, TdT, TPMT, TRG, TRP1, TSTA, Folate receptor alpha, L1-CAM, CAIX, gpA33, GD3, GM2, VEGFR, Intergrins, carbohydrates, IGFIR, EPHA3, TRAILR1, TRAILR2, RANKL, FAP, TGF-beta, hyaluronic acid, collagen, tenascin C, and tenascin W.

Exemplary embodiments of the systems, methods and computer-accessible medium according to the present disclosure can be provided to analyze on a computer system a feature of a medical image of a tissue and a visual feature of a histopathology sample of the tissue using a specific procedure, where the specific procedure can be or include a machine-learning procedure trained on a set of images of cancerous anatomies.

The systems, methods and computer-accessible medium according to certain exemplary embodiments of the present disclosure can generate and/or analyze the images of cancerous anatomies can be magnetic resonance images. For example, the cancerous anatomies can be cancerous breasts, and/or the images of cancerous anatomies can be magnetic resonance images of cancerous breasts.

With the exemplary embodiments of the systems, methods and computer-accessible medium according to the present disclosure, e.g., prior to the analyzing, it is possible to perform an imaging procedure on the tissue to obtain the medical image of the tissue.

### Exemplary AI System

The exemplary systems, methods and computer-accessible medium according to the present disclosure can provide and/or utilize an AI system which can implement a machine learning procedure (e.g., a neural network) that can learn to predict risk of cancer recurrence, diagnose cancer or cancer subtype, or predict treatment response. In various exemplary embodiments of the systems, methods and computer-accessible medium according to the present disclosure, the treatment can be a therapeutic regimen of an agent that is therapeutically effective for the target cancer, for example, compounds described herein, and others as are appropriate for a patient's particular circumstances. In further exemplary embodiments of the systems, methods and computer-accessible medium according to the present disclosure, it is possible to predict the likelihood that a patient having breast cancer will experience a relapse or recurrence. For example, the computer-implemented system can be an artificial intelligence (AI) system, and/or the exemplary machine-learning procedure can be trained on a set of images of cancerous anatomies.

Using the exemplary embodiments of the systems, methods and computer-accessible medium according to the present disclosure, it is possible to:
(a) process a medical image analysis system, whereas the medical image analysis system can comprise:
   (1) an image receiving portal,
   (2) a first feature extractor,
   (3) a histopathology data receiving portal,
   (4) a second feature extractor, and
   (5) an output function;
(b) receive by the image receiving portal a medical image of a tissue from an image input source;
(c) extract by the first feature extractor at least one feature from the medical image of the tissue to provide an extracted image feature;
(d) receive by the histopathology data receiving portal digitized histopathology data of the tissue from a histopathology data source;
e) extract by the second feature extractor at least one feature from the digitized histopathology data of the tissue to provide an extracted histopathology feature;
(f) process the extracted image feature and the extracted histopathology feature to provide a multi-modal representation of the tissue;
(g) process the multimodal representation of the tissue with a specific procedure to provide a prediction regarding a medical outcome of the tissue; and
(h) output by the output function the prediction regarding the medical outcome of the tissue.

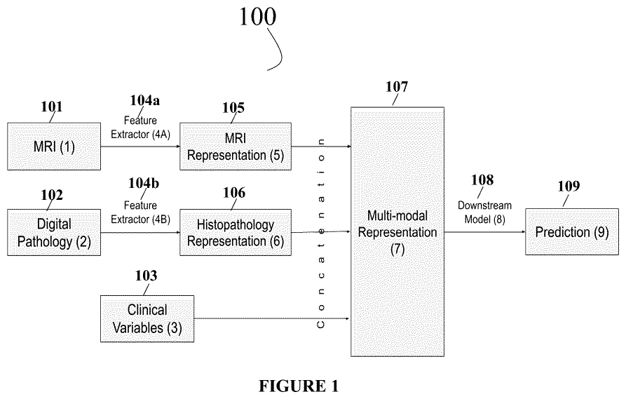

FIG. 1 illustrates an exemplary diagram of a machine learning system 100 for generating predictions about breast cancer outcomes. The exemplary system 100 comprises input modalities comprising an image receiving portal 101, a histopathology data receiving portal 102, and a clinical variables receiving portal 103, a first feature extractor 104a, a second feature 104b, extracted MRI representation 105, extracted digitized histopathology data representation 106, multi-modal representation 107, downstream model 108, and prediction 109. The inputs 101 102 103 comprise breast magnetic resonance imaging (MRI) 101, digital pathology 102, and clinical variables 103.

Using the exemplary embodiments of the systems, methods and computer-accessible medium according to the present disclosure, the outcome can be a medical outcome.

With the exemplary embodiments of the systems, methods and computer-accessible medium according to the present disclosure, the medical outcome can be a cancer diagnosis, cancer staging, cancer recurrence, response to treatment, treatment benefit, prognosis, survival rate, or a combination thereof.

Using the exemplary embodiments of the systems, methods and computer-accessible medium according to the present disclosure, the medical outcome can be a duration of time to breast cancer relapse, survival rate, patient age at the time of surgery, tumor stage, tumor size, tumor location (e.g., unifocal, multifocal), number of positive nodes, or surgery type, status of one or more biomarker(s), tumor grade, or histological type, or a combination thereof. In exemplary embodiments of the systems, methods and computer-accessible medium according to the present disclosure, duration of time to breast cancer relapse or prognosis can be assessed by, for example, overall survival, invasive disease-free survival (iDFS), distant disease-free survival (dDFS), metastasis-free interval (MFI), or a combination thereof.

With the exemplary embodiments of the systems, methods and computer-accessible medium according to the present disclosure, the overall survival can be the time from diagnosis of a specific disease, e.g., breast cancer, to death from any cause. Overall survival rate can be to the percentage of subjects who are alive at a certain time after diagnosis, among all subjects diagnosed with a specific disease, e.g., breast cancer.

Using the exemplary embodiments of the systems, methods and computer-accessible medium according to the present disclosure, the invasive disease-free survival (iDFS) in the context of breast cancer can be the time from diagnosis of breast cancer to occurrence of any of the following: ipsilateral invasive breast cancer recurrence, regional invasive breast cancer recurrence, distant recurrence, death attributable to any cause, contralateral invasive breast cancer, second nonbreast invasive cancer, or a combination thereof. For example, the distant disease-free survival (dDFS) in the context of breast cancer can be the time from diagnosis to relapse at a distant site or death from any cause. In addition or alternatively, metastasis-free interval (MFI) in the context of breast cancer can be the time from the diagnosis of primary nonmetastatic breast cancer to the date of the first distant metastases.

According to the exemplary embodiments of the systems, methods and computer-accessible medium of the present disclosure, the input modality can be data from an imaging tool. Non-limiting examples of imaging tools include ultrasound, magnetic resonance imaging (MRI), functional MRI (fMRI), dynamic contrast-enhanced MRI (DCE-MRI), breast MRI, cardiac MRI, computed tomography (CT), X-ray, mammography, positron emission tomography (PET), single photon emission computed tomography (SPECT), fluoroscopy, or a combination thereof.

In certain exemplary embodiments of the systems, methods and computer-accessible medium according to the present disclosure, the breast MRI 101 can be MRI images from cancerous breasts. For example, the breast MRI 101 can be from a breast cancer patient prior to treatment. The breast MRI 101 can contain at least one pre-contrast T1-weighted sequence. The breast MRI 101 can contain at least two post-contrast T1-weighted sequence.

### Exemplary Input Modality

In exemplary embodiments of the systems, methods and computer-accessible medium according to the present disclosure, the input modality can be from a section of histology or histopathology specimen. For example, the digital pathology can be digitized histopathology slides of breast cancer specimens.

Histopathology includes the microscopic examination of specimens, e.g., tissues, obtained or otherwise derived from a subject, e.g., a patient, to assess a disease state. Histopathology specimens can result from processing the specimen, e.g., tissue, in a manner that affixes the specimen, or a portion thereof, to a microscope slide. For example, thin sections of a tissue specimen can be obtained using a microtome or any suitable device, and the thin sections can be affixed to a slide. Any suitable specimen affixation methods can be used. Non-limiting examples of affixed specimen include formalin-fixed, paraffin-embedded tissue (FFPE), flash frozen tissue using dry ice or liquid nitrogen, cryopreserved tissue, and zinc-fixed tissue.

In further exemplary embodiments of the systems, methods and computer-accessible medium according to the present disclosure, the tissue can be human breast tissue and/or a human body part that can be in need of inspection for possible disease, a human body part that is in need of inspection for possible cancer, a human breast that is in need of inspection for possible breast cancer, or a human breast that is afflicted with breast cancer.

The specimen can be further processed, for example, by applying a stain to assist in visualization. Any suitable stains for visualizing cells and tissues can be used. Non-limiting examples of the stains include Haemotoxylin and Eosin (H&E), methylene blue, Masson's trichome, Congo red, Oil Red O, silver nitrate, melanin, Gomori trichrome, Mallory trichrome, Alcian blue, Crystal violet, toluidine blue, and safranin.

The stain can visualize a specific biomarker(s). A biomarker(s) can be a protein, DNA, or RNA biomarker. Any suitable labeling methods can be used to visualize the biomarkers. Staining techniques that depend on the use of labeled detection reagents that specifically bind to a marker of interest, such as immunofluorescence, immunohistochemistry, in situ hybridization, RNAScope can be used.

In exemplary embodiments of the systems, methods and computer-accessible medium according to the present disclosure, the clinical variables can comprise categorical and numerical variables that contain information about the subject, e.g., a patient and the condition or disease. Non-limiting examples of clinical variables can include tumor stage, tumor grade, patient's age at diagnosis, histological subtype, patient's age at the time of surgery, menopausal status, number of positive nodes (N+), number of nodules, surgery type, and molecular biomarker status. Non-limiting examples of biomarkers include estrogen (ER), progesterone (PR), HER-2, BRCA1, BRCA2, TP53, Ki-67, and a combination thereof. The molecular biomarker status can be the presence or absence of one or more mutations (i.e., BRCA1 or BRCA2). The molecular biomarker status can be gene expression level (i.e., mRNA level).

In yet further exemplary embodiments of the systems, methods and computer-accessible medium according to the present disclosure, molecular biomarker status can be determined using qPCR. The molecular biomarker status can be determined using ELISA, Sanger sequencing, microarray, and/or next-generation sequence (NGS). Non-limiting examples of NGS includes whole-genome sequencing (WGS), whole-exome sequencing (WES), RNA sequencing, ATAC sequencing, bisulfite sequencing, and chromatin immunoprecipitation (ChIP) sequencing. Further or alternatively, the molecular biomarker can be an epigenetic marker and/or a plurality of biomarkers.

In exemplary embodiments of the systems, methods and computer-accessible medium according to the present disclosure, the clinical variables can be in the form of clinical electronic health record (EHR) data. The EHR data can be stored in a software. Non-limiting examples of EHR software include enterprise EHR software, software as a service (SaaS) EHR, custom EHR builds, on-site EHR data storage, EHR data remotely hosted on dedicated servers, cloud-based EHR data storage, certified EHR, stand-alone EHR, or integrated EHR and EPM systems.

In exemplary embodiments of the systems, methods and computer-accessible medium according to the present disclosure, the information from the electronic health record can be a tumor stage of the tissue and/or a status of a molecular biomarker in a subject that provided the tissue.

Exemplary embodiments of the systems, methods and computer-accessible medium according to the present disclosure can comprise 1, 2, 3 or more input modalities, or at most 1, 2, or 3 input modalities. For example, the inputs can be the same or different input modality. In exemplary embodiments of the systems, methods and computer-accessible medium according to the present disclosure, the input modality can be patient outcomes.

### Exemplary Feature Extractor

Exemplary embodiments of the systems, methods and computer-accessible medium according to the present disclosure can comprise at least one, two, or three feature extractors. Alternatively, the exemplary embodiments of the systems, methods and computer-accessible medium according to the present disclosure can comprise at most one, two, or three feature extractors.

In exemplary embodiments of the systems, methods and computer-accessible medium according to the present disclosure, the feature extractor can take various forms. Non-limiting examples of a feature extractor include: convolutional neural networks; recurrent neural networks; autoencoders; transformer networks; and ensembles thereof. The feature extractor can be used to extract information from the input modalities, such as from imaging data or digital pathology. In exemplary embodiments of the systems, methods and computer-accessible medium according to the present disclosure, the feature extractor can extract at least one feature from a medical image of a tissue to provide an extracted image feature to provide an extracted histopathology feature. The feature extractor can extract at least one feature from digitized histopathology data of a tissue. For example, as shown in FIG. 1, a feature extractor 104a can be provided for extracting information from MRI. In addition, a feature extractor 104b can be provided for extracting information from digital pathology. In exemplary embodiments of the systems, methods and computer-accessible medium according to the present disclosure, the convolutional neural network can be a ResNet architecture of convolutional neural network.

In exemplary embodiments of the systems, methods and computer-accessible medium according to the present disclosure, the medical image can be a magnetic resonance image of a human body part that is in need of inspection for possible disease, a magnetic resonance image of a human body part that is in need of inspection for possible cancer, a magnetic resonance image of a human breast that is in need of inspection for possible breast cancer, or a magnetic resonance image of a human breast that is afflicted with breast cancer. The extracted image feature can be a visual detail that suggests presence of a cancer.

In certain exemplary embodiments of the systems, methods and computer-accessible medium according to the present disclosure, the digitized histopathology data of the tissue can be or include an image of a digitized histopathology slide of the tissue, an image of a digitized histopathology slide of a human breast, an image of a digitized histopathology slide of a human body part that is in need of inspection for possible disease, an image of a digitized histopathology slide of a human body part that is in need of inspection for possible cancer, an image of a digitized histopathology slide of a human breast that is in need of inspection for possible breast cancer, or an image of a digitized histopathology slide of a human breast that is afflicted with breast cancer. For example, the extracted histopathology feature can be a visual detail that suggests presence of a cancer.

### Exemplary Data Representation

After extracting information by the feature extractor 104a and the feature extractor 104b, the machine learning system 100, according to exemplary embodiments of the systems, methods and computer-accessible medium according to the present disclosure, can generate a data representation. For example, the data representation can be low-dimensional representations of the input modality generated by the feature extractor feature extractor 104a and feature extractor 104b. Further or alternatively, the MRI representation 105 and/or the histopathology representation 106 can be low-dimensional representations generated by the feature extractor 104a and feature extractor 104b, respectively.

### Exemplary Multi-Modal Representation

In exemplary embodiments of the systems, methods and computer-accessible medium according to the present disclosure, the multi-modal representation 107 can be a combined data structure derived from multiple input modalities. The representation can be a mathematical vector, matrix, or tensor. Non-limiting examples of input modalities include: mathematical vectors; histopathology representations; and clinical variables. The multi-modal representation 107 can be a mathematical vector that is a result of concatenating MRI Representation 105, Histopathology Representation, 106 and clinical variables 103. The input modalities can be combined by a combining operation. Non-limiting examples of a combining operation include methods such as: concatenation; element-wise addition; element-wise multiplication; weighted combinations; statistical fusion methods; deep-learning-based multi-modal fusion; transformer-based fusion; autoencoder fusion; and any other machine learning or statistical learning method suitable for combining different representations.

### Exemplary Downstream Model

For example, the downstream model 108 can be trained to generate predictions 109 using the multi-modal representation 107. The downstream model 108 can be a trained, machine-learning procedure. The downstream model 108 can use various forms of machine learning approaches. Non-limiting examples of machine learning approaches include gradient boosting, a neural network, and ensembles thereof. Gradient boosting is a machine learning technique used to construct an ensemble of simpler models, such as decision trees, to optimize and improve the overall prediction accuracy. The downstream model 108 can iteratively improve each new model based on the mistakes made by previous ones, thereby reducing the bias and variance of the final model. Non-limiting examples of gradient boosting techniques include Catboost or XGBoost. The neural network described herein can comprise multiple fully connected layers.

### Exemplary Predictions

Predictions 109 made by the system 100 can be in the form of probabilities comprising floating point values. The floating point values can be in a range between 0 and 1, where O is a least likely probability and 1 is a most likely probability. For example, the floating point value can be about 0, about 0.05, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, and/or about 1.

In exemplary embodiments of the systems, methods and computer-accessible medium according to the present disclosure, the predictions 109 can be probabilities of breast cancer recurrence, e.g., at different time horizons. For example, the time horizons can be in the range of 3-10 years, about 6 months, about 2 years, about 3 years, about 4 years, about 5 years, about 6 years, about 7 years, about 8 years, about 9 years, and/or about 10 years.

In exemplary embodiments of the systems, methods and computer-accessible medium according to the present disclosure, the predictions 109 can be predictions of possible therapies on impacting the probability of cancer recurrence,

regarding the medical outcome, e.g., of the tissue can be a likelihood of a relapse of a disease, a likelihood of a relapse of cancer, a likelihood of a relapse of breast cancer, a likelihood of favorable response to a therapy for a condition present in the tissue, a likelihood of favorable response to a therapy for cancer present in the tissue, or a likelihood of favorable response to a therapy for breast cancer present in the tissue.

### Exemplary Training and Evaluating the AI System

The exemplary embodiments of systems, methods and computer-accessible medium according to the present disclosure can be provided for training the AI system. For example, the AI system can comprise a machine learning procedure, such as a neural network, and/or can be a trained machine learning procedure. The AI system can be a network.

The exemplary AI system as described herein can be configured to undergo at least one training phase, whereas the machine learning software module can be trained to carry out one or more tasks including data extraction, data analysis, and generation of output, and/or using a two-stage training process, comprising, e.g.:
1. a pretraining feature extractors stage; and/or
2. a training the Downstream Model stage.

In exemplary embodiments of the systems, methods and computer-accessible medium according to the present disclosure, during the pretraining feature extractor stage, the feature extractors can be pre-trained on an initial task to generate meaningful representations of the input data, e.g., MRI, digital pathology, clinical variables, auxiliary clinical variables, or patient outcomes. The task can be supervised or self-supervised.

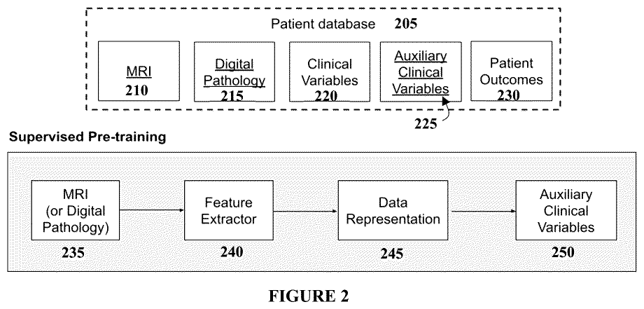

FIG. 2 illustrates an exemplary diagram of supervised pre-training. For example, supervised pre-training can include a patient database 205, comprising MRI data 210, digital pathology 215, clinical variables 220, auxiliary clinical variables 225, and patient outcomes 230. It can also include MRI data 235, feature extractor 240, data representation 245, and auxiliary clinical variables 250. In exemplary embodiments of the systems, methods and computer-accessible medium according to the present disclosure, a supervised pretraining can comprise, e.g.:
a. Providing feature extractors with labeled input-output pairs, such as MRI and information about cancer status; and
b. The network learning to map input data to corresponding output labels following standard optimization specific procedures used for convolutional neural networks.

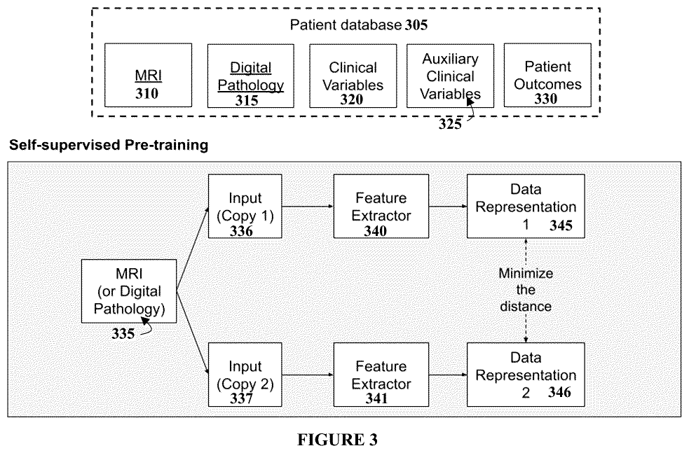

FIG. 3 illustrates an exemplary diagram of self-supervised pretraining, which can include, e.g., a patient database 305, comprising MRI data 310, digital pathology 315, clinical variables 320, auxiliary clinical variables 325, and patient outcomes 330. It can also include MRI data 335, an input copy 1 336, and input copy 2 337, a first feature extractor 340, a second feature extractor 341, data representation 1 345, and data representation 2 346. In exemplary embodiments of the systems, methods and computer-accessible medium according to the present disclosure, a self-supervised pretraining comprises feature extractors 340 and 341 learning to generate useful representations 345 and 346 of the input data 336 and 337 without relying on explicitly labeled data. A proxy task can be designed, wherein the network captures meaningful features in the data. For example, a network can be taught to minimize the distance between two Data Representations 345 and 346 generated by slightly modifying the input.

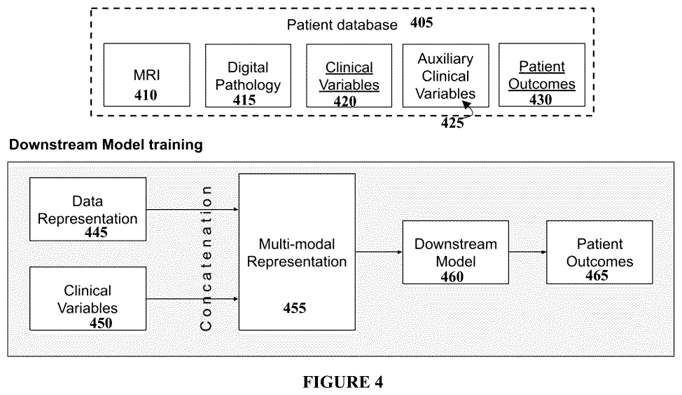

FIG. 4 illustrates an exemplary diagram of Downstream Model training. Downstream Model training can include a patient database 405, comprising MRI data 410, digital pathology 415, clinical variables 420, auxiliary clinical variables 425, and patient outcomes 430. It can also include data representation 445, clinical variables 450, multi-modal representation 455, downstream model 460, and patient outcomes 465. In exemplary embodiments of the systems, methods and computer-accessible medium according to the present disclosure, during the training the Downstream Model stage, the Downstream Model 460 can be trained on the target downstream task using Data Representations generated by pre-trained feature extractors.

In various exemplary embodiments of the systems, methods and computer-accessible medium according to the present disclosure, to generate Data Representations from feature extractors, the final layer (i.e., the classification layer) can be removed, and the low-dimensional feature representation in the penultimate layer is saved (Data Representation). Input to the Downstream Model 460 can comprise concatenated low-dimensional representations of MRI, Digital Pathology and Clinical Variables. Downstream Model 460 can use a plurality of input modalities and learn to map the input modalities to target output 465, such as a probability of breast cancer risk recurrence.

According to additional exemplary embodiments of the systems, methods and computer-accessible medium according to the present disclosure, various tasks can be optimized by the Downstream Model 460. The Downstream Model 460 can learn to classify the probability of breast cancer risk recurrence within a pre-specified number of years, and/or to optimize a time-to-event model (i.e., a survival analysis model), such as Cox proportional hazards, AFT (Accelerated Failure Time), or discrete time model.

For example, the Downstream Model 460 can determine that the subject is at risk of a breast cancer recurrence of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The Downstream Model 460 can determine that the subject is at risk of a breast cancer recurrence at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

### Exemplary Treatment Response Prediction

In exemplary embodiments of the systems, methods and computer-accessible medium according to the present disclosure, the exemplary AI system can be trained to predict treatment benefit or response. For example, the AI system can compare two or more available treatment options for which the subject is eligible. Further or alternatively, the AI system can predict whether a therapy can be successful, how long the therapy can take, or be used to determine whether a new therapy is necessary. For example, the supervising physician can be directed to administer a treatment option based at least in part on the outcome of the treatment response prediction. Non-limiting examples of treatment options include all those discussed herein.

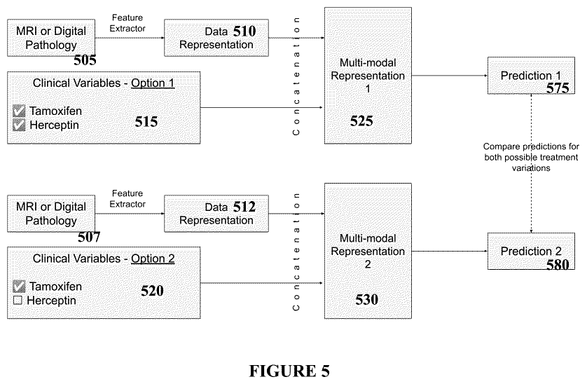

FIG. 5 illustrates an exemplary diagram of treatment response prediction. In exemplary embodiments of the systems, methods and computer-accessible medium according to the present disclosure, the exemplary treatment response prediction can be trained by modifying Clinical Variables 515, 520 as input to the system 525, 530 (along with data representation 510, 512 extracted from MRI data 505, 507). For example, the AI System according to exemplary embodiments of the systems, methods and computer-accessible medium according to the present disclosure can be trained using Clinical Variables 515, 520 that contain information about a patient's treatment (e.g., whether or not the patient has received a particular type of treatment (e.g., chemotherapy, hormone therapy, or radiation), specific drug (e.g., trastuzumab or tamoxifen), or length or dosage of a particular treatment regimen).

In exemplary embodiments of the systems, methods and computer-accessible medium according to the present disclosure, the input Clinical Variables 515, 520 can be manipulated so that the Clinical variables 515, 520 represent different treatment options. For example, treatment option 1, 515 can be tamoxifen and Herceptin, while treatment option 2, 520 can be tamoxifen alone. Multiple modified Inputs can be processed through the AI System, and a Prediction 575, 580 can be made. Then, Predictions, 575, 580 can be compared across various treatment options, revealing which Input modification is most or least likely to yield desired patient outcomes.

In exemplary embodiments of the systems, methods and computer-accessible medium according to the present disclosure, the AI system can optimize therapies for the subject. For example, any suitable causal inference techniques can be used to allow the AI system to make causal inferences.

Exemplary embodiments of the systems, methods and computer-accessible medium according to the present disclosure can be used by healthcare providers (e.g., primarily medical oncologists, radiation oncologists, or breast surgeons) to make more informed decisions about treatment.

In exemplary embodiments of the systems, methods and computer-accessible medium according to the present disclosure, the exemplary AI system can be used to escalate or de-escalate treatment (i.e., chemotherapy, hormone therapy, immunotherapy, biologics, radiation therapy, breast surgery). An exemplary escalation or de-escalation can include adding or removing a drug to/from the systemic treatment regimen; shortening the duration of treatment; changing the type of treatment; or lowering or increasing the dosage of a drug.

In exemplary embodiments of the systems, methods and computer-accessible medium according to the present disclosure, the prediction regarding a medical outcome of the tissue can be a likelihood of favorable response to a therapy for cancer present in the tissue. For example, the prediction regarding a medical outcome of the tissue can be a likelihood of favorable response to a therapy for breast cancer present in the tissue.

The exemplary embodiments of the systems, methods and computer-accessible medium according to the present disclosure can also comprise administering to a subject that provided the sample a therapeutic intervention that corresponds to the prediction regarding the medical outcome of the tissue.

In exemplary embodiments of the systems, methods and computer-accessible medium according to the present disclosure, the AI system can be used to determine whether a specific drug (such as a chemotherapeutic agent) or type of treatment is suitable for a patient. For example, the AI system can be used to determine patient population with HER2-positive breast cancer that can benefit from 6-months versus 12-months trastuzumab treatment; patient population with HER2-positive breast cancer that can benefit from trastuzumab treatment alone versus trastuzumab together with pertuzumab, and patient population planned for chemotherapy treatment that can benefit from the addition of anthracyclines in the regimen.

For example, the AI System can be used to predict the response of a chemotherapeutic agent. Non-limiting examples of chemotherapeutic agents include: 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™ RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASER), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), improsulfan, ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), piposulfan, procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

The AI System can be used to predict the response of a biologic. Biologics useful in the treatment of cancers and a binding molecule as described herein can be administered, for example, in conjunction with such known biologics. Non-limiting examples for treatment of breast cancer include: HERCEPTIN® (trastuzumab); FASLODEX® (fulvestrant); ARIMIDEX® (anastrozole); Aromasin® (exemestane); FEMARA® (letrozole); and NOLVADEX® (tamoxifen). Other biologics with which the binding molecules as described herein can be combined include: AVASTIN® (bevacizumab); and ZEVALIN® (ibritumomab tiuxetan).

Non-limiting examples of biologics for the treatment of colorectal cancer include: AVASTIN®; ERBITUX® (cetuximab); GLEEVEC® (imatinib mesylate); and ERGAMISOL® (levamisole hydrochloride). Non-limiting examples for the treatment of lung cancer include: TARCEVA® (erlotinib HCL). Non-limiting examples for the treatment of multiple myeloma include: VELCADE® (bortezomib). Additional biologics include THALIDOMID® (thalidomide).

In some exemplary embodiments, the AI System can be used to predict the response of a cancer therapeutic antibody. Non-limiting examples of cancer therapeutic antibodies include: 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVABR), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDER), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

Additional non-limiting examples of cancer therapeutic agents include: alkylating agents (e.g., thiotepa and cyclosphosphamide), alkyl sulfonates (e.g., busulfan, improsulfan, and piposulfan), aziridines (e.g., such as benzodopa, carboquone, meturedopa, and uredopa), ethylenimines and methylamelamines (e.g., altretamine, triethylenemelamine, trietylenephosphoramide, triethiylenethiophosphoramide, and trimethylolomelamine), acetogenins (e.g., bullatacin and bullatacinone), camptothecin (e.g., topotecan); bryostatin; callystatin, CC-1065 (e.g., adozelesin, carzelesin and bizelesin), cryptophycins (e.g., cryptophycin 1 and cryptophycin 8), dolastatin, duocarmycin (e.g., KW-2189 and CB1-TM1); eleutherobin, pancratistatin, sarcodictyin, spongistatin, nitrogen mustards (e.g., chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard) nitrosureas (e.g., carmustine, chlorozotocin, fotemustine, lomustine, nimustine, and ranimnustine), aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (e.g., morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin, anti-metabolites (e.g., methotrexate and 5-fluorouracil (5-FU)), folic acid analogues (e.g., denopterin, pteropterin, and trimetrexate), purine analogs (e.g., fludarabine, 6-mercaptopurine, thiamiprine, and thioguanine) pyrimidine analogs (e.g., ancitabine, azacitidine, 6-azauridine, carmofur, cytarabine, dideoxyuridine, doxifluridine, enocitabine, and floxuridine); androgens (e.g., calusterone, dromostanolone propionate, epitiostanol, mepitiostane, and testolactone), anti-adrenals (e.g., as mitotane and trilostane), folic acid replenisher (e.g., frolinic acid; aceglatone; aldophosphamide glycoside), aminolevulinic acid, eniluracil, amsacrine, bestrabucil, bisantrene, edatrexate, defofamine, demecolcine, diaziquone, elformithine, elliptinium acetate, epothilone, etoglucid, gallium nitrate, hydroxyurea, lentinan, lonidainine, maytansinoids (e.g., such as maytansine and ansamitocins), mitoguazone, mitoxantrone, mopidanmol, nitraerine, pentostatin, phenamet, pirarubicin, losoxantrone, podophyllinic acid, 2-ethylhydrazide, procarbazine, PSKpolysaccharide complex, razoxane, rhizoxin, sizofiran, spirogermanium, tenuazonic acid, triaziquone, 2,2′, 2″-trichlorotriethylamine, trichothecenes (e.g., T-2 toxin, verracurin A, roridin A and anguidine), urethan, vindesine, dacarbazine, mannomustine, mitobronitol, mitolactol, pipobroman, gacytosine, arabinoside (e.g., Ara-C), cyclophosphamide; toxoids (e.g., paclitaxel and docetaxel), gemcitabine, 6-thioguanine, mercaptopurine, platinum coordination complexes (cisplatin, oxaliplatin, and carboplatin), vinblastine, platinum, etoposide (e.g., VP-16), ifosfamide, mitoxantrone, vincristine, vinorelbine, novantrone, teniposide, edatrexate, daunomycin, aminopterin, xeloda, ibandronate, irinotecan (e.g., CPT-11), topoisomerase inhibitor, difluorometlhylornithine (DMFO), retinoids (retinoic acid), capecitabine, carboplatin, procarbazine, and plicamycin.

In exemplary embodiments of the systems, methods and computer-accessible medium according to the present disclosure, the AI System can determine that the treatment response at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

### Exemplary Web Application Interface

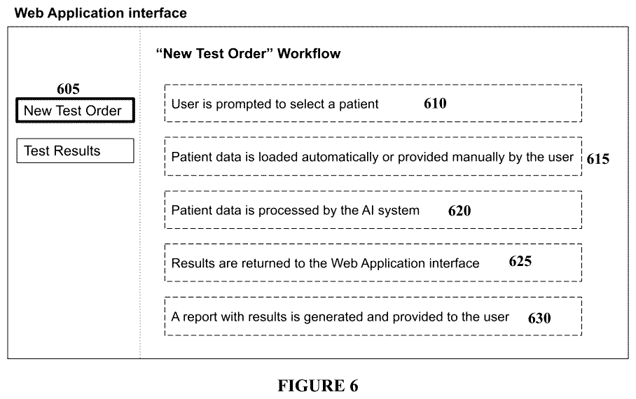

In exemplary embodiments of the systems, methods and computer-accessible medium according to the present disclosure, the exemplary AI System described herein can be implemented using a Web Application interface. FIG. 6 illustrates an exemplary diagram of Web Application interface workflow. For example, a user of the Web Application can make orders for a new test 605. A user can be prompted to select a patient at 610. Then, patient data can be internally loaded at 615 and processed through the AI system at 620. The results may be returned at 625, and finally, the user can be presented with a report with all results at 630. The report can use a standardized template.

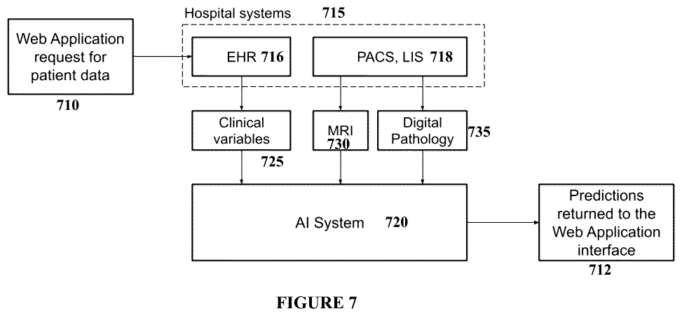

For example, the exemplary AI System described herein can be implemented with the Web Application and a hospital system. FIG. 7 illustrates an example integration between the Web Application (comprising Web Application request for patient data 710 and predictions returned to the Web Application 712), hospital systems 715, and the AI System 720. The hospital system 715 can comprise electronic health records (EHR) 716, picture archive and communication system (PACS), or laboratory information system (LIS) 718. In exemplary embodiments of the systems, methods and computer-accessible medium according to the present disclosure, the input modalities described herein can be stored in the EHR 716, PACS, or LIS 718. The Clinical Variables 725 can be stored in the EHR 716. The MRI data 730 or the Digital Pathology 735 can be stored in the PACS or LIS 718.

### Exemplary Machine Learning

The exemplary embodiments of the systems, methods and computer-accessible medium according to the present disclosure can provide AI systems comprising a machine learning procedure (e.g., a neural network) that can learn to predict risk of cancer recurrence, diagnose cancer or cancer subtype, or predict treatment response.

The exemplary AI system of exemplary embodiments of the systems, methods and computer-accessible medium according to the present disclosure can use a feature extractor to extract information from any one of the input modalities, such as MRI, imaging data, Digital Pathology, Clinical Variables, Auxiliary Clinical Variables, Patient Outcomes, or a combination thereof. The exemplary feature extractor can be trained using data from any one or a plurality of the input modalities.

### Exemplary Training Phase

A machine learning procedure utilized and/or included in the systems, methods and computer-accessible medium according to exemplary embodiments of the present disclosure can be configured to undergo at least one training phase wherein the machine learning software module can be trained to carry out one or more tasks including data extraction, data analysis, and generation of output.

In exemplary embodiments of the systems, methods and computer-accessible medium according to the present disclosure, the machine learning procedure can be trained using a data set and a target in a manner of supervised learning. the exemplary data set can be divided into a training set, a test set, and, in some exemplary embodiments, a validation set. A target can be specified that contains the correct classification of each input value in the data set. For example, data from an input modality can be repeatedly presented to the machine learning procedure, and for each sample presented during training, the output generated by the machine learning procedure can be compared with the desired target. The difference between the target and the set of input samples can be calculated, and the machine learning procedure can be modified to cause the output to more closely approximate the desired target value. A back-propagation procedure can be utilized to cause the output to more closely approximate the desired target value. After several training iterations, the machine learning procedure output can closely match the desired target for each sample in the input training set. Subsequently, when new input data, not used during training, is presented to the machine learning procedure, the specific procedure can generate an output classification value indicating into which of the categories the new sample is most likely to fall. The machine learning procedure can generalize from the training to interpret new, previously unseen input samples. This exemplary feature of a machine learning procedure according to exemplary embodiments of the systems, methods and computer-accessible medium according to the present disclosure, allows classification of almost any input data that has a mathematically formulatable relationship to the category to which the data should be assigned.

In exemplary embodiments of the systems, methods and computer-accessible medium according to the present disclosure, the machine learning procedure can utilize an individual learning model. An individual learning model can be based on the machine learning procedure having trained on data from a single individual and thus, the machine learning procedure that utilizes an individual learning model can be configured to be used on a single individual on whose data the module was trained.

In addition or alternatively, the machine learning procedure can utilize a global training model. A global training model can be based on the machine learning procedure having trained on data from multiple individuals and thus, a machine learning procedure that utilizes a global training model can be configured to be used on multiple individuals.

In exemplary embodiments of the systems, methods and computer-accessible medium according to the present disclosure, the machine learning procedure can utilize a simulated training model. A simulated training model is based on the machine learning procedure having trained on data from the input modalities.

Unsupervised learning can be used, in exemplary embodiments of the systems, methods and computer-accessible medium according to the present disclosure, to train a machine learning procedure to use input data such as, for example, MRI data and output, for example, a risk of cancer recurrence. Unsupervised learning, in exemplary embodiments of the systems, methods and computer-accessible medium according to the present disclosure, can include a feature extraction, which can be performed by the machine learning module on the input data. Extracted features can be used for visualization, for classification, for subsequent supervised training, and more generally for representing the input for subsequent storage or analysis. For example, each training case can comprise of a plurality of input modalities.

Machine learning procedure that are suitable for unsupervised training include k-means clustering, mixtures of multinomial distributions, affinity propagation, discrete factor analysis, hidden Markov models, Boltzmann machines, restricted Boltzmann machines, autoencoders, convolutional autoencoders, recurrent neural network autoencoders, and long short-term memory autoencoders.

The exemplary machine learning procedure can include a training phase and a prediction phase. The training phase can provide data to train the machine learning procedure. Non-limiting examples of types of data inputted into a machine learning software module for training can include medical image data, clinical data (e.g., from a health record), clinical variables, auxiliary clinical variables, encoded data, encoded features, and metrics derived from input modalities. Data that are inputted into the machine learning procedure can be used, in the exemplary embodiments of the systems, methods and computer-accessible medium according to the present disclosure, to construct a hypothesis function to determine the risk of cancer recurrence.

In exemplary embodiments of the systems, methods and computer-accessible medium according to the present disclosure, a machine learning procedure can be configured to determine whether the outcome of the hypothesis function was achieved and based on that analysis to determine with respect to the data upon which the hypothesis function was constructed. That is, the outcome may tend to either reinforce the hypothesis function with respect to the data upon which the hypothesis functions were constructed or to contradict the hypothesis function with respect to the data upon which the hypothesis function was constructed. In certain exemplary embodiments of the systems, methods and computer-accessible medium according to the present disclosure, depending on how close the outcome tends to be to an outcome determined by the hypothesis function, the machine learning procedure can adopt, adjust, or abandon the hypothesis function with respect to the data upon which the hypothesis function was constructed. As such, the machine learning procedure of exemplary embodiments of the systems, methods and computer-accessible medium according to the present disclosure can dynamically learn through the training phase what characteristics of an input (e.g., data) are most predictive in determining whether the features of a subject's recorded input modalities are predictive of a particular outcome.

### Exemplary Prediction Phase

Following training, the machine learning procedure of exemplary embodiments of the systems, methods and computer-accessible medium according to the present disclosure can be used to determine, for example, the risk of cancer recurrence.

The prediction phase can use the constructed and optimized hypothesis function from the training phase to predict the risk of cancer recurrence. In exemplary embodiments of the systems, methods and computer-accessible medium according to the present disclosure, in the prediction phase, the machine learning procedure can be used to analyze data derived from the input modalities independent of any system or device described herein.

The exemplary probability threshold can be used in conjunction with a final probability to determine whether a given recording matches the trained prediction. Alternatively or in addition, the probability threshold can be used to tune the sensitivity of the trained network. For example, the probability threshold can be 1%, 2%, 5%, 10%, 15%, 20%, 25%, 30%, 35%, 40%, 45%, 50%, 55%, 60%, 65%, 70%, 75%, 80%, 85%, 90%, 95%, 98%, or 99%. The probability threshold can be adjusted if the accuracy, sensitivity, or specificity falls below a predefined adjustment threshold. The adjustment threshold can be used to determine the parameters of the training period. For example, if the accuracy of the probability threshold falls below the adjustment threshold, the system can extend the training period and/or require additional data from the input modalities. Additional data from the input modalities can be included into the training data, and/or can be used to refine the training data set.

### Exemplary Machine Learning Techniques

The exemplary embodiments of the systems, methods and computer-accessible medium according to the present disclosure can provide various machine learning (ML) techniques. ML can involve identifying and recognizing patterns in existing data to facilitate making predictions for subsequent data. ML can include a ML model for example, a ML procedure. Machine learning, whether analytical or statistical, can provide deductive or abductive inference based on real or simulated data. The ML model can be a trained model. ML techniques can comprise one or more supervised, semi-supervised, self-supervised, or unsupervised ML techniques. For example, a ML model can be a trained model that is trained through supervised learning (e.g., various parameters are determined as weights or scaling factors).

ML can comprise one or more of regression analysis, regularization, classification, dimensionality reduction, ensemble learning, meta learning, association rule learning, cluster analysis, anomaly detection, deep learning, or ultra-deep learning. Non-limiting examples of ML include: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, generative adversarial networks, vision transformers, long short-term memory networks (LSTM), and masked autoencoders.

### Exemplary Training

Training the ML model of can include, in exemplary embodiments of the systems, methods and computer-accessible medium according to the present disclosure, selecting one or more untrained data models to train using a training data set. The selected untrained data models can include any type of untrained ML models for supervised, semi-supervised, self-supervised, or unsupervised machine learning. The selected, untrained data models can be specified based on input (e.g., user input) specifying relevant parameters to use as predicted variables or other variables to use as potential explanatory variables. For example, the selected, untrained data models can be specified to generate an output (e.g., a prediction) based upon the input. Conditions for training the ML model from the selected untrained data models can likewise be selected, such as limits on the ML model complexity or limits on the ML model refinement past a certain point. The ML model can be trained (e.g., via a computer system such as a server) using the training data set. In exemplary embodiments of the systems, methods and computer-accessible medium according to the present disclosure, a first subset of the training data set can be selected to train the ML model. The selected, untrained data models can then be trained on the first subset of training data set using appropriate ML techniques, based upon the type of ML model selected and any conditions specified for training the ML model. In exemplary embodiments of the systems, methods and computer-accessible medium according to the present disclosure, due to the processing power requirements of training the ML model, the selected untrained data models can be trained using additional computing resources (e.g., cloud computing resources). Such training can continue, in exemplary embodiments of the systems, methods and computer-accessible medium according to the present disclosure, until at least one aspect of the ML model is validated and meets selection criteria to be used as a predictive model.

### Exemplary Validation

In exemplary embodiments of the systems, methods and computer-accessible medium according to the present disclosure, one or more aspects of the ML model can be validated using a second subset of the training data set (e.g., distinct from the first subset of the training data set) to determine accuracy and robustness of the ML model. Such validation can include applying the ML model to the second subset of the training data set to make predictions derived from the second subset of the training data. The ML model can then be evaluated to determine whether performance is sufficient based upon the derived predictions. The sufficiency criteria applied to the ML model can vary depending upon the size of the training data set available for training, the performance of previous iterations of trained models, or user-specified performance requirements. If the ML model does not achieve sufficient performance, additional training can be performed. Additional training can include refinement of the ML model or retraining on a different first subset of the training dataset, after which the new ML model can again be validated and assessed. When the ML model has achieved sufficient performance, in exemplary embodiments of the systems, methods and computer-accessible medium according to the present disclosure, the ML can be stored for present or future use. The ML model can be stored as sets of parameter values or weights for analysis of further input (e.g., further relevant parameters to use as further predicted variables, further explanatory variables, further user interaction data, etc.), which can also include analysis logic or indications of model validity in some instances. In exemplary embodiments of the systems, methods and computer-accessible medium according to the present disclosure, a plurality of ML models can be stored for generating predictions under different sets of input data conditions. For example, the ML model can be stored in a database (e.g., associated with a server).

### Exemplary Deep Learning

Exemplary embodiments of the systems, methods and computer-accessible medium according to the present disclosure can implement one or more deep-learning techniques. Deep learning is an example of machine learning (ML) that can be based on a set of procedures that model high-level abstractions in data by using multiple processing layers, with complex structures or otherwise, composed of multiple non-linear transformations. In exemplary embodiments of the systems, methods and computer-accessible medium according to the present disclosure, a drop out method can be used to reduce overfitting. At each training stage, individual nodes can either be dropped out of the net (e.g., ignored) with probability 1-p or kept with probability p, so that a reduced network is left. Incoming and outgoing edges to a dropped-out node can also be removed. In exemplary embodiments of the systems, methods and computer-accessible medium according to the present disclosure, the reduced network can be trained on the data in that stage. The removed nodes can then be reinserted into the network with the original weights.

### Exemplary Decision Tree

Exemplary embodiments of the systems, methods and computer-accessible medium according to the present disclosure can implement one or more decision tree or random forest techniques. A decision tree can be a supervised ML procedure that can be applied to both regression and classification problems. Decision trees can mimic the decision-making process of a human brain. For example, a decision tree can grow from a root (base condition), and when the tree meets a condition (internal node/feature), the tree splits into multiple branches. The end of the branch that does not split anymore is an outcome (leaf). A decision tree can be generated using a training data set according to the following operations: (1) Starting from a root node (the entire dataset), the procedure can split the dataset in two branches using a decision rule or branching criterion, (2) each of the two branches can generate a new child node, (3) for each new child node, the branching process can be repeated until the dataset cannot be split any further, and/or (4) each branching criterion can be chosen to maximize information gain (e.g., a quantification of how much a branching criterion reduces a quantification of how mixed the labels are in the children nodes). The exemplary labels can be the data or the classification that is predicted by the decision tree.

### Exemplary Random Forest

A random forest regression is an extension of the decision tree model that tends to yield more robust predictions by stretching the use of the training data partition. Whereas a decision tree can make a single pass through the data, a random forest regression can bootstrap 50% of the data (e.g., with replacement) and build many trees. Rather than using all explanatory variables as candidates for splitting, a random subset of candidate variables can be used for splitting to produce trees that have different data and different variables. The predictions from the trees, collectively referred to as the forest, are then averaged to produce the final prediction. Many trees (e.g., one hundred trees) can be included in a random forest model, with a number (e.g., 3, 6, 10, etc.) of terms sampled per split, a minimum of number (e.g., 1, 2, 4, 10, etc.) of splits per tree, and a minimum split size (e.g., 16, 32, 64, 128, 256, etc.). Random forests can be trained in a similar way as decision trees. Training a random forest can include the following operations: (1) select randomly k features from the total number of features; (2) create a decision tree from these k features using the same operations as for generating a decision tree; and (3) repeat the previous two operations until a target number of trees is created.

### Exemplary Long Short-Term Memory (LSTM)

Exemplary embodiments of the systems, methods and computer-accessible medium according to the present disclosure can implement one or more long short-term memory (LSTM) techniques. LSTM can be an artificial neural network used in the fields of artificial intelligence and deep learning. Unlike standard feedforward neural networks, LSTM can use feedback connections. The LSTM architecture can provide a short-term memory for a recurrent neural network (RNN). Such RNN can process not only single data points (such as images), but also entire sequences of data (such as speech or video). The connection weights and biases in the RNN can change once per episode of training, analogously to how physiological changes in synaptic strengths store long-term memories. The activation patterns in the network can change once per time-step, analogously to how the moment-to-moment change in electric firing patterns in the brain store short-term memories. The LSTM architecture can provide a short-term memory for a RNN that can last many (e.g., thousands) timesteps.

In exemplary embodiments of the systems, methods and computer-accessible medium according to the present disclosure, a LSTM unit can comprise a cell, an input gate, an output gate, and a forget gate. The exemplary cell can remember values over arbitrary time intervals and the input gate, the output gate, and the forget gate can regulate the flow of information into and out of the cell. Forget gates can be used to decide what information to discard from a previous state by assigning a previous state, compared to a current input, a value between 0 and 1 (e.g., a (rounded) value of 1 can mean to keep the information, and a value of 0 means to discard the information). The input gate can decide which pieces of new information to store in the current state, using the same system as the forget gates. The output gate can control which pieces of information in the current state to output (e.g., by assigning a value from 0 to 1 to the information, considering the previous and current states). Selectively outputting relevant information from the current state can allow that the LSTM network maintains useful, long-term dependencies to make predictions, both in current and future time-steps. LSTM networks can be well-suited to classifying, processing, and making predictions based on time series data, since lags of unknown duration between important events in a time series can occur. LSTMs can resolve the vanishing gradient problem that can be encountered when training traditional RNNs. Relative insensitivity to gap length can be an advantage of LSTM over RNNs, hidden Markov models, and other sequence learning methods in numerous applications.

In certain exemplary embodiments of the systems, methods and computer-accessible medium according to the present disclosure, LSTMs can be used with one or more various types of neural networks (e.g., convolutional neural networks (CNNs), deep neural network (DNNs), recurrent neural networks (RNNs), etc.). In exemplary embodiments of the systems, methods and computer-accessible medium according to the present disclosure, CNNs, LSTM, and DNNs can be complementary in modeling capabilities and can be combined in a unified architecture. For example, in such unified architecture, CNNs can be well-suited at reducing frequency variations, LSTMs can be well-suited at temporal modeling, and DNNs can be well-suited for mapping features to a more separable space. For example, input features to a ML model using LSTM techniques in the unified architecture can include segment features for each of a plurality of segments. To process the input features for each of the plurality of segments, the segment features for the segment can be processed using one or more CNN layers to generate first features for the segment. The first features can be processed using one or more LSTM layers to generate second features for the segment. The second features can be processed using one or more fully connected neural network layers to generate third features for the segments, where the third features can be used for classification operations.

According to certain exemplary embodiments of the present disclosure, to process the first features using the one or more LSTM layers to generate the second features, the first features can be processed using a linear layer to generate reduced features having a reduced dimension from a dimension of the first features. The reduced features can be processed using the one or more LSTM layers to generate the second features. Short-term features having a first number of contextual frames can be generated based on the input features, where features generated using the one or more CNN layers can include long-term features having a second number of contextual frames that are more than the first number of contextual frames of the short-term features. In exemplary embodiments of the systems, methods and computer-accessible medium according to the present disclosure, the one or more CNN layers, the one or more LSTM layers, and the one or more fully connected neural network layers can be jointly trained to determine trained values of parameters of the one or more CNN layers, the one or more LSTM layers, and the one or more fully connected neural network layers. In some exemplary embodiments, the input features include log-mel features having multiple dimensions. The input features include one or more contextual frames indicating a temporal context of a signal (e.g., input data). Implementations for such unified architecture can leverage complementary advantages associated with each of a CNN, LSTM, and DNN. For example, convolutional layers can reduce spectral variation in input and help the modeling of LSTM layers. Having DNN layers after LSTM layers can help reduce variation in the hidden states of the LSTM layers. Training the unified architecture jointly can provide a better overall performance. Training in the unified architecture can also remove the need to have separate CNN, LSTM and DNN architectures. By adding multi-scale information into the unified architecture, information can be captured at different time scales.

### Exemplary Support Vector Machines

The exemplary embodiments of the systems, methods and computer-accessible medium according to the present disclosure can implement one or more support vector machine learning techniques. In machine learning, support vector machines (SVMs) can be supervised learning models with associated learning procedures that analyze data for classification and regression analysis. SVMs can be a robust prediction method, being based on statistical learning. SVMs can be well-suited for domains characterized by the existence of large amounts of data, noisy patterns, or the absence of general theories.

SVMs can map input vectors into high dimensional feature space through non-linear mapping function, chosen a priori. In this high dimensional feature space, an optimal separating hyperplane can be constructed. The optimal hyperplane can then be used to determine things such as class separations, regression fit, or accuracy in density estimation. More formally, a SVM constructs a hyperplane or set of hyperplanes in a high or infinite-dimensional space, which can be used for classification, regression, or other tasks like outlier detection.

Support vectors can be defined as the data points that lie closest to the decision surface (or hyperplane). Support vectors can therefore be the data points that are most difficult to classify and can have direct bearing on the optimum location of the decision surface. Given a set of training examples, each marked as belonging to one of two categories, a SVM training procedure can build a model that assigns new examples to one category or the other, making the procedure a non-probabilistic binary linear classifier. SVM can map training examples to points in space to maximize the width of the gap between the two categories. New examples can then be mapped into that same space and predicted to belong to a category based on which side of the gap the examples fall. In addition to performing linear classification, SVMs can efficiently perform a non-linear classification using what is called the kernel trick, implicitly mapping inputs into high-dimensional feature spaces.

Within a support vector machine, the dimensionally of the feature space can be large. For example, a fourth-degree polynomial mapping function can cause a 200-dimensional input space to be mapped into a 1.6 billionth dimensional feature space. SVMs assist in discovering knowledge from vast amounts of input data.

### Exemplary Gradient Boosting

Exemplary embodiments of the systems, methods and computer-accessible medium according to the present disclosure can implement one or more gradient boosting techniques. Gradient boosting is a machine learning technique used in regression and classification tasks, among others. Gradient boosting gives a prediction model in the form of an ensemble of weak prediction models, which are typically decision trees. When a decision tree is the weak learner, the resulting procedure is called gradient-boosted trees. A gradient-boosted trees model is built in a stage-wise fashion as in other boosting methods, but generalizes the other methods by allowing optimization of an arbitrary differentiable loss function.

Exemplary k-Nearest Neighbors

The exemplary embodiments of the systems, methods and computer-accessible medium according to the present disclosure can implement one or more K-nearest neighbors (KNN) techniques. KNN is a non-parametric classification method. In KNN classification, the output is a class membership. An object is classified by a plurality vote of neighbors, with the object being assigned to the class most common among the k nearest neighbors (k is a positive integer, typically small). If k=1, then the object is assigned to the class of that single nearest neighbor. In KNN regression, the output is the property value for the object. This value is the average of the values of k nearest neighbors. KNN is a type of classification wherein the function is approximated locally, and computation is deferred until function evaluation. Since this exemplary procedure relies on distance for classification, if the features represent different physical units or come in vastly different scales, then normalizing the training data can improve accuracy.

The exemplary embodiments of the systems, methods and computer-accessible medium according to the present disclosure can implement one or more Monte Carlo techniques. Monte Carlo is a broad class of computational procedures that rely on repeated random sampling to obtain numerical results. The underlying concept is to use randomness to solve problems that might be deterministic in principle.

### Exemplary One-Hot Encoding

The exemplary embodiments of the systems, methods and computer-accessible medium according to the present disclosure can implement one or more one-hot encoding techniques. One-hot encoding can be used to deal with categorical data. For example, a ML model can use input variables that are numeric. The categorical variables can be transformed in a pre-processing part. Categorical data can be either nominal or ordinal. Ordinal data can have a ranked order of values and can therefore be converted to numerical data through ordinal encoding.

### Exemplary Processing Units

The exemplary embodiments of the systems, methods and computer-accessible medium according to the present disclosure can provide computer systems or processing units that are programmed to implement methods of the disclosure. A computer system or processing unit can be programmed or otherwise configured to, for example, (i) extract features from any one or a plurality of input modalities, (ii) train and test a trained procedure, (iii) generate data representations using the trained procedure, (iv) concatenate data representations into a multi-modal representation, (v) train and test a Downstream Model, and (vi) generate a prediction.

The exemplary processing unit can regulate various aspects of analysis, calculation, and/or generation of the present disclosure. The processing unit can be or include an electronic device of a user or a computer system that is remotely located with respect to the electronic device. The exemplary electronic device can be a mobile electronic device.

The exemplary processing unit can include a central processing unit, which can be a single core or multi-core processor, or a plurality of processors for parallel processing. The processing unit can also include memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface, and peripheral devices can be in communication with the CPU through a communication bus, such as a motherboard. The storage unit can be a data storage unit (or data repository) for storing data. The processing unit can be operatively coupled to a computer network with the aid of the communication interface. The network can be the Internet, an internet and/or extranet, or an intranet and/or extranet that is in communication with the Internet.

The network—in some exemplary cases—can be or include a telecommunication or data network. The network can include one or more computer servers, which can provide distributed computing, such as cloud computing. For example, one or more computer servers may enable cloud computing over the network to perform various aspects of analysis, calculation, and generation of the present disclosure, such as, for example, (i) extract features from any one or a plurality of input modalities, (ii) train and test a trained procedure, (iii) generate data representations using the trained procedure, (iv) concatenate data representations into a multi-modal representation, (v) train and test a Downstream Model, and (vi) generate a prediction. Such cloud computing can be provided by cloud computing platforms such as, for example, Amazon Web Services (AWS), Microsoft Azure, Google Cloud Platform, and IBM cloud. The network, in some cases, with the aid of the processing unit, can implement a peer-to-peer network, which can permit that devices coupled to the processing unit behave as a client or a server.

The CPU can comprise one or more computer processors or one or more graphics processing units (GPUs). The CPU can execute a sequence of machine-readable instructions, which can be embodied in a program or software. The instructions can be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the processing unit can be included in the circuit. In some cases, the circuit is an application specific integrated circuit (ASIC).

A storage unit can be used store files, such as drivers, libraries, and saved programs. The storage unit can store user data, e.g., user preferences and user programs. The processing unit in some cases can include one or more additional data storage units that are external to the computer system, such as located on a remote server that is in communication with the processing unit through an intranet or the Internet.

The exemplary computer system can communicate with one or more remote processing units through the network. For instance, the processing unit can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iphone, Android-enabled device, Blackberry®), or personal digital assistants.

Exemplary embodiments of the systems, methods and computer-accessible medium according to the present disclosure can be implemented by way of machine (e.g., computer processor) executable code stored on an electronic storage location of the computer system, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some exemplary embodiments, the electronic storage unit can be precluded, and machine-executable instructions are stored on memory.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of exemplary embodiments of the systems, methods and computer-accessible medium according to the present disclosure, such as the computer system or processing unit, can be embodied in programming. Various aspects of the technology can be provided as executable code and/or associated data that is carried on or embodied in a type of machine readable medium. Machine-executable code can be stored on an electronic storage unit, such as memory (e.g., read-only memory, random-access memory, flash memory) or a hard disk. Storage type media can include any or all the tangible memory of the computers, processors, or associated modules thereof, such as various semiconductor memories, tape drives, and disk drives, which can provide non-transitory storage at any time for the software programming. All or portions of the software can at times be communicated through the Internet or various other telecommunication networks. Such communications, for example, can allow loading of the software from one computer or processor into another, for example, from a management server or host computer into the computer platform of an application server. Thus, another type of media that can bear the software elements includes optical, electrical, and electromagnetic waves, such as used across physical interfaces between local devices, through wired and optical landline networks and over various air-links. The physical elements that carry such waves, such as wired or wireless links or optical links, also can be considered as media bearing the software.

An exemplary machine readable medium, such as computer-executable code, can take many forms, including but not limited to, a tangible storage medium, a carrier wave medium or physical transmission medium. Non-volatile storage media include, for example, optical or magnetic disks, such as any of the storage devices in any computer(s), such as can be used to implement databases, etc., Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code or data. Many of these forms of computer readable media can be involved in carrying one or more sequences of one or more instructions to a processor for execution.

The processing unit described herein can include or be in communication with an electronic display that comprises a user interface (UI) for providing, for example, (i) a visual display indicative of training and testing of a trained procedure, (ii) a visual display of data indicative of a liver disease state of a subject, (iii) a quantitative measure of a liver disease state of a subject, (iv) an identification of a subject as having a liver disease state, or (v) an electronic report indicative of the liver disease state of the subject. Examples of UIs include, without limitation, a graphical user interface (GUI) and web-based user interface.

Methods and systems of the present disclosure can be implemented by way of one or more exemplary procedures. The exemplary procedure can be implemented by way of software upon execution by the central processing unit. The exemplary procedure can, for example, (i) extract features from any one or a plurality of input modalities, (ii) train and test a trained alg procedure, (iii) generate data representations using the trained procedure, (iv) concatenate data representations into a multi-modal representation, (v) train and test a Downstream Model, and (vi) generate a prediction.

TABLE 1
Summary

| | Training set | Validation set | Test set | Total |
|---|---|---|---|---|
| Examinations | 14,198 | 3403 | 3936 | 21,537 |
| Patients | 8679 | 2142 | 2642 | 13,463 |
| Age, mean years (SD) | 54.56 ± 13.2 | 54.30 ± 13.0 | 55.23 ± 12.7 | 54.64 ± 13.1 |

Race

| | | | | |
|---|---|---|---|---|
| White | 9819 (69.2) | 2317 (68.1) | 2738 (69.6) | 14,874 (69.1) |
| Black | 802 (5.6) | 205 (6.0) | 244 (6.2) | 1251 (5.8) |
| Asian | 549 (3.9) | 182 (5.3) | 164 (4.2) | 895 (4.2) |
| Other/unknown | 3028 (21.3) | 699 (20.5) | 790 (20.1) | 4517 (21.0) |

Diagnosis

| | | | | |
|---|---|---|---|---|
| Malignant | 2337 (16.5) | 582 (17.1) | 861 (21.9) | 3780 (17.6) |
| Benign | 3380 (23.8) | 804 (23.6) | 1148 (29.2) | 5332 (24.8) |
| Negative | 10,040 (70.7) | 2397 (70.4) | 2491 (63.3) | 14,928 (69.3) |

BI-RADS category

| | | | | |
|---|---|---|---|---|
| BI-RADS 0 | 446 (3.1) | 103 (3.0) | 102 (2.6) | 651 (3.0) |
| BI-RADS 1 | 2213 (15.6) | 469 (13.8) | 596 (15.1) | 3278 (15.2) |
| BI-RADS 2 | 5818 (41.0) | 1416 (41.6) | 1378 (35.0) | 8612 (40.0) |
| BI-RADS 3 | 1292 (9.1) | 331 (9.7) | 333 (8.5) | 1956 (9.1) |
| BI-RADS 4 | 2601 (18.3) | 667 (19.6) | 956 (24.3) | 4224 (19.6) |
| BI-RADS 5 | 94 (0.7) | 27 (0.8) | 40 (1.0) | 161 (0.7) |
| BI-RADS 6 | 1205 (8.5) | 298 (8.8) | 385 (9.8) | 1888 (8.8) |
| Unknown | 529 (3.7) | 92 (2.7) | 146 (3.7) | 767 (3.6) |

Background parenchymal enhancement

| | | | | |
|---|---|---|---|---|
| Minimal/mild | 8933 (62.9) | 2117 (62.2) | 2498 (63.5) | 13,548 (62.9) |
| Moderate/marked | 3309 (23.3) | 858 (25.2) | 1068 (27.1) | 5235 (24.3) |
| Unknown | 1956 (13.8) | 428 (12.6) | 370 (9.4) | 2754 (12.8) |

Fibroglandular tissue

| | | | | |
|---|---|---|---|---|
| A/B (fatty/scattered) | 4664 (32.8) | 1094 (32.1) | 1304 (33.1) | 7062 (32.8) |
| C/D (heterogeneous/ | 7295 (51.4) | 1796 (52.8) | 2178 (55.3) | 11,269 (52.3) |
| extreme) | | | | |
| Unknown | 2239 (15.8) | 513 (15.1) | 454 (11.5) | 3206 (14.9) |

TABLE 2
Summary of histological and molecular cancer subtypes. Values are n
(% of all malignant cases), reported on an exam level. HR, hormone
receptor; ER, estrogen receptor; PR, progesterone receptor; HER2,
human epidermal growth factor receptor 2. Many examinations in
the dataset had missing information about HER2 receptor status
and/or Ki-67 status. Such examinations only have partial
luminal/nonluminal classification. For example, if cancer was
classified as ER+/PR+ but the information about HER-2 status
was missing, then the cancer was considered “luminal, unknown.”

| | | | |
|---|---|---|---|
| Training | Validation | Test | |
| set | set | set | Total |

Histological type

| | | | | |
|---|---|---|---|---|
| Ductal carcinoma in situ | 1485 | 389 | 570 | 2444 |
| Invasive ductal carcinoma | 1404 | 347 | 523 | 2274 |
| Metastatic carcinoma | 417 | 82 | 138 | 637 |
| Adenocarcinoma | 272 | 55 | 106 | 433 |
| Invasive lobular carcinoma | 263 | 55 | 87 | 405 |
| Invasive mammary carcinoma | 86 | 18 | 33 | 137 |
| Papillary carcinoma | 51 | 11 | 5 | 67 |
| Other/unknown | 33 | 8 | 12 | 53 |
| Lymphoma (B cell/ | 2 | 2 | 3 | 7 |
| lymphocytic) | | | | |

Molecular type
Luminal (HR⁺)

| | | | | |
|---|---|---|---|---|
| Luminal A | 911 | 222 | 326 | 1459 |
| Luminal B | 237 | 62 | 78 | 377 |
| Luminal, unknown* | 539 | 141 | 202 | 882 |

Nonluminal (HR⁻)

| | | | | |
|---|---|---|---|---|
| Triple negative | 177 | 36 | 63 | 276 |
| HER2 enriched | 56 | 19 | 21 | 96 |
| Nonluminal, unknown* | 147 | 38 | 55 | 240 |
| Unknown | 270 | 64 | 116 | 450 |

TABLE 3
Summary of the extractor feature performance. Results
are presented with 95% CIs (bootstrapping with
N = 2000 replicates), unless indicated otherwise.

| Dataset | AUC ROC | AUC PR |
|---|---|---|
| N | 0.924 (0.915-0.933) | 0.720 (0.689-0.751) |
| J | 0.797 (0.756-0.838) | 0.596 (0.522-0.674) |
| D | 0.969 (0.960-0.976) | 0.977 (0.971-0.982) |
| T | 0.966 (0.942-0.985) | 0.973 (0.954-0.988) |
| N Reader study set | | |
| Standalone feature | 0.924 (0.880-0.962) | 0.784 (0.656-0.887) |
| extractor system | | |
| Radiologists, average | 0.890 [0.850-0.948] | 0.758 [0.712-0.868] |
| [range] | | |
| J reader study set | | |
| Standalone feature | 0.802 (0.712-0.881) | 0.558 (0.407-0.731) |
| extractor system | | |
| Radiologists, average | 0.818 [0.787-0.849] | 0.683 [0.667-0.699] |
| [range] | | |

TABLE 4
MRI manufacturer and model used for data sets. MRI scanners were sorted by number
of total cases in the data set, descending. An empty cell indicates that the
specific data set did not contain any cases acquired on the machine.

| Manufacturer/ | | | Number of cases | | | |
|---|---|---|---|---|---|---|
| [symbol] | Magnet | N | J | D | T | Total |
| Siemens Symphony | 1.5 T | 9,638 | | | 2 | 9,640 |
| Siemens Trio Tim | 3 T | 8,142 | | 58 | | 8,200 |
| Siemens Skyra | 3 T | 1,940 | 1 | 57 | | 1,998 |
| Siemens Espree | 1.5 T | 668 | | | 1 | 669 |
| Philips Achieva | 1.5 T | 477 | | | 16 | 493 |
| Siemens | 1.5 T | | 392 | | | 392 |
| [symbol] | | | | | | |
| Siemens Avanto | 1.5 T | 132 | 1 | 179 | 3 | 315 |
| GE SIGNA HDx | 1.5 T | | | 272 | 8 | 280 |
| GE SIGNA HDxt | 1.5 T | | | 248 | 6 | 254 |
| Siemens Verio | 3 T | 175 | | | | 175 |
| GE SIGNA HDe | 1.5 T | 112 | | | | 112 |
| Siemens Aera | 1.5 T | 72 | | | | 72 |
| Siemens Verio Dot | 3 T | 65 | | | | 65 |
| Siemens | 3 T | 64 | | | | 64 |
| [symbol] | | | | | | |
| Hitachi ECHELON | 1.5 T | 24 | | | | 24 |
| GE Optima MR450w | 1.5 T | | | 98 | | 98 |
| GE SIGNA EXCITE | 1.5 T | | | 10 | 85 | 95 |
| Siemens Sonata | 3 T | | | | 9 | 9 |
| GE DISCOVERY MR750 | 3 T | | | | 1 | 1 |
| Unknown | — | 28 | | | | 28 |
| | | 21,537 | 394 | 922 | 131 | 22,984 |

[symbol]

indicates data missing or illegible when filed

TABLE 5
Exemplary Results of the reader study on the N subset, reported with 95% confidence intervals estimated with
bootstrap (N = 2,000). An average performance across all 5 readers was reported. Average reader performance
was calculated as a simple mean of metrics for all readers. To calculate sensitivity and specificity for
readers, BI-RADS 4 was used as a binarization threshold. Studies classified by radiologists as BI-RADS 4
or 5 were considered as positive and BI-RADS 1, 2, 3 as negative. For AI predictions, a decision threshold
was selected such that the AI system's sensitivity closely matches average reader sensitivity.

| Reader | AUROC | AUPRC | Sensitivity | Specificity | PPV | NPV |
|---|---|---|---|---|---|---|
| Reader 1 | 0.850 | 0.712 | 0.780 | 0.786 | 0.485 | 0.933 |
| | (0.779-0.917) | (0.567-0.833) | (0.659-0.895) | (0.721-0.849) | (0.381-0.593) | (0.883-0.972) |
| Reader 2 | 0.860 | 0.715 | 0.854 | 0.660 | 0.393 | 0.946 |
| | (0.780-0.935) | (0.556-0.853) | (0.737-0.969) | (0.582-0.745) | (0.294-0.490) | (0.904-0.991) |
| Reader 3 | 0.948 | 0.868 | 0.976 | 0.704 | 0.460 | 0.991 |
| | (0.908-0.978) | (0.778-0.941) | (0.913-1.000) | (0.634-0.764) | (0.366-0.556) | (0.971-1.000) |
| Reader 4 | 0.916 | 0.775 | 0.976 | 0.610 | 0.392 | 0.990 |
| | (0.866-0.954) | (0.640-0.867) | (0.917-1.000) | (0.536-0.679) | (0.291-0.487) | (0.965-1.000) |
| Reader 5 | 0.873 | 0.721 | 0.854 | 0.761 | 0.479 | 0.953 |
| | (0.820-0.932) | (0.596-0.842) | (0.750-0.949) | (0.700-0.822) | (0.370-0.582) | (0.915-0.985) |
| Avg Reader | 0.890 | 0.758 | 0.888 | 0.704 | 0.442 | 0.962 |
| AI System | 0.924 | 0.784 | 0.897 | 0.796 | 0.517 | 0.969 |
| | | (0.656- [symbol] | | | | |

[symbol]

indicates data missing or illegible when filed

TABLE 6
Results of the secondary reader study on the J subset reported with 95% confidence intervals estimated
with bootstrap (N = 2,000). Average reader performance was calculated as a simple mean of
metrics for all readers. To calculate sensitivity and specificity for readers, BI-RADS 4 was used
as a binarization threshold. That is, studies classified by radiologists as BI-RADS 4 or 5 were
considered as positive and BI-RADS 1, 2, 3 as negative. For AI predictions, a decision threshold
was selected such that the AI system's sensitivity closely matches average reader sensitivity.

| Reader | AUROC | AUPRC | Sensitivity | Specificity | PPV | NPV |
|---|---|---|---|---|---|---|
| Reader | 0.787 | 0.667 | 0.690 | 0.816 | 0.508 | 0.905 |
| 1 | (0.708- | (0.515-0.786) | (0.548-0.816) | (0.747-0.873) | (0.380-0.635) | (0.853-0.949) |
| | 0.859) | | | | | |
| Reader | 0.849 | 0.699 | 0.834 | 0.717 | 0.449 | 0.939 |
| 2 | (0.776- | (0.559-0.827) | (0.719-0.938) | (0.665-0.791) | (0.338-0.562) | (0.894-0.982) |
| | 0.916) | | | | | |
| Avg | 0.818 | 0.683 | 0.762 | 0.767 | 0.479 | 0.922 |
| Reader | | | | | | |
| AI | 0.802 | 0.558 | 0.762 | 0.762 | 0.469 | 0.921 |
| System | (0.712- | (0.407-0.731) | (0.622-0.886) | (0.695-0.828) | (0.353-0.589) | (0.870-0.966) |
| | 0.881) | | | | | |

TABLE 7
Inter-reader variability analysis results. To calculate κ, the predictions were
binarized (made by both AI and readers on the N reader study cases) into positive and
negative classes. Binarization threshold was set at 2% [probability of malignancy]
according to the clinical guidelines. Readers were asked to follow these guidelines.
Readers were required to assign a probability of >2% to any BI-RADS 4 or 5 prediction.

| | | Fleiss' κ | Avg | |
|---|---|---|---|---|
| Category | Hybrid type/Description | [symbol] | [symbol] | AUROC s2 |
| No hybrid | Interreader variability between readers | 0.5567 | 0.890 | 1.7 × 10⁻³ |
| | | (0.50-0.63) | | |
| AI hybrid | Unweighted average of reader prediction | 0.77 | 0.939 | 2.1 × 10⁻⁴ |
| | and AI prediction | (0.72-0.82) | | |
| Baseline 1 | Unweighted average of reader prediction | 0.5608 | 0.890 | 1.7 × 10⁻³ |
| | [symbol] | [symbol] | | |
| Baseline 2 | [symbol] | [symbol] | [symbol] | [symbol] |

[symbol]

indicates data missing or illegible when filed

TABLE 8
Subgroup performance. Reported values are n (95% confidence intervals). Confidence intervals were calculated with a bootstrap (2,000 replicates).
PPV, positive predictive value; NPV, negative predictive value. As no malignant examples were found in BI-RADS 1 and 2 categories in the
test set, AUROC would not be defined for those groups. BI-RADS 1 and 2 were combined with BI-RADS 3 to generate the results. For AI predictions,
a decision threshold was selected such that the AI system's sensitivity closely matched average reader sensitivity.

| | | | | | | | |
|---|---|---|---|---|---|---|---|
| Group | n | AUROC | AUPRC | Sensitivity | Specificity | PPV | NPV |

BI-RADS risk assessment category

| | | | | | | | |
|---|---|---|---|---|---|---|---|
| BIRADS 1/2/3 | 2,307 | 0.84 (0.68-0.97) | 0.09(0.01-0.27) | 0.75 (0.46-1.00) | 0.83 (0.82-0.84) | 0.01 (0.00-0.02) | 1.00 (1.00-1.00) |
| BIRADS 4 | 956 | 0.87 (0.85-0.89) | 0.72(0.67-0.76) | 0.88 (0.85-0.91) | 0.61 (0.58-0.63) | 0.42 (0.39-0.45) | 0.94 (0.93-0.96) |
| BIRADS 5 | 40 | 0.87 (0.78-0.95) | 0.89(0.78-0.97) | 0.91 (0.82-0.98) | 0.47 (0.31-0.64) | 0.68 (0.56-0.79) | 0.81 (0.64-0.95) |
| BIRADS 6 | 385 | 0.90 (0.87-0.92) | 0.88(0.85-0.92) | 0.90 (0.86-0.93) | 0.67 (0.62-0.72) | 0.70 (0.65-0.74) | 0.88 (0.85-0.92) |
| BIRADS 0 | 102 | 0.94 (0.88-0.98) | 0.61(0.35-0.84) | 0.94 (0.82-1.00) | 0.75 (0.68-0.81) | 0.27 (0.16-0.38) | 0.99 (0.98-1.00) |
| unknown | 146 | 0.92 (0.87-0.96) | 0.75(0.61-0.85) | 0.89 (0.81-0.98) | 0.79 (0.74-0.84) | 0.45 (0.35-0.55) | 0.97 (0.95-0.99) |

Patient age at the time of examination

| | | | | | | | |
|---|---|---|---|---|---|---|---|
| Age <40 | 399 | 0.91 (0.87-0.94) | 0.65(0.53-0.76) | 0.89 (0.81-0.95) | 0.74 (0.70-0.77) | 0.27 (0.22-0.33) | 0.98 (0.97-0.99) |
| Age <50 | 1,294 | 0.91 (0.89-0.93) | 0.67(0.61-0.73) | 0.89 (0.85-0.92) | 0.73 (0.71-0.75) | 0.30 (0.27-0.33) | 0.98 (0.97-0.99) |
| Age ≥50 | 2,642 | 0.93 (0.92-0.94) | 0.74(0.71-0.78) | 0.89 (0.86-0.91) | 0.79 (0.78-0.80) | 0.37 (0.35-0.40) | 0.98 (0.98-0.99) |

Breast cancer histological subtype

| | | | | | | | |
|---|---|---|---|---|---|---|---|
| DCIS | 570 | 0.91 (0.89-0.92) | 0.93(0.92-0.95) | 0.89 (0.86-0.91) | 0.68 (0.64-0.72) | 0.76 (0.72-0.79) | 0.84 (0.80-0.87) |
| IDC | 523 | 0.93 (0.92-0.95) | 0.95(0.94-0.96) | 0.92 (0.90-0.94) | 0.64 (0.60-0.68) | 0.74 (0.71-0.78) | 0.88 (0.84-0.91) |
| Meta | 138 | 0.96 (0.93-0.98) | 0.97(0.94-0.99) | 0.96 (0.93-0.99) | 0.58 (0.49-0.66) | 0.72 (0.66-0.79) | 0.93 (0.86-0.98) |
| Adenoca | 106 | 0.95 (0.92-0.98) | 0.96(0.93-0.98) | 0.98 (0.95-1.00) | 0.55 (0.45-0.65) | 0.72 (0.65-0.79) | 0.96 (0.91-1.00) |
| ILC | 87 | 0.90 (0.85-0.94) | 0.94(0.90-0.96) | 0.86 (0.79-0.92) | 0.63 (0.52-0.74) | 0.75 (0.67-0.83) | 0.77 (0.67-0.87) |
| IMC | 33 | 0.94 (0.88-0.99) | 0.95(0.89-0.99) | 0.94 (0.85-1.00) | 0.75 (0.59-0.90) | 0.80 (0.67-0.92) | 0.92 (0.81-1.00) |
| Other/unknown | 20 | 0.84 (0.70-0.96) | 0.91(0.79-0.98) | 0.81 (0.63-0.95) | 0.53 (0.29-0.75) | 0.65 (0.46-0.83) | 0.71 (0.45-0.93) |

Breast cancer molecular subtype

| | | | | | | | |
|---|---|---|---|---|---|---|---|
| Luminal A | 326 | 0.93 (0.90-0.94) | 0.95(0.93-0.96) | 0.91 (0.88-0.94) | 0.68 (0.63-0.73) | 0.77 (0.73-0.81) | 0.86 (0.82-0.90) |
| Luminal B | 78 | 0.96 (0.92-0.99) | 0.97(0.94-0.99) | 0.96 (0.91-1.00) | 0.67 (0.56-0.78) | 0.76 (0.67-0.84) | 0.94 (0.87-1.00) |
| Triple negative | 63 | 0.93 (0.87-0.97) | 0.95(0.91-0.98) | 0.91 (0.82-0.97) | 0.71 (0.59-0.83) | 0.76 (0.66-0.86) | 0.88 (0.78-0.96) |
| HER2-enriched | 21 | 0.97 (0.91-1.00) | 0.98(0.93-1.00) | 0.95 (0.85-1.00) | 0.67 (0.45-0.85) | 0.74 (0.57-0.89) | 0.93 (0.79-1.00) |

Background parenchymal enhancement

| | | | | | | | |
|---|---|---|---|---|---|---|---|
| Minimal | 884 | 0.94 (0.92-0.96) | 0.78(0.71-0.84) | 0.89 (0.84-0.94) | 0.85 (0.83-0.86) | 0.35 (0.30-0.39) | 0.99 (0.98-0.99) |
| Mild | 1,614 | 0.93 (0.91-0.94) | 0.72(0.68-0.77) | 0.89 (0.86-0.92) | 0.79 (0.77-0.80) | 0.37 (0.34-0.40) | 0.98 (0.97-0.99) |
| Moderate | 884 | 0.91 (0.88-0.93) | 0.71(0.65-0.77) | 0.88 (0.84-0.92) | 0.68 (0.66-0.71) | 0.32 (0.28-0.35) | 0.97 (0.96-0.98) |
| Marked | 184 | 0.87 (0.82-0.92) | 0.66(0.54-0.77) | 0.92 (0.85-0.98) | 0.60 (0.54-0.65) | 0.33 (0.27-0.40) | 0.97 (0.95-0.99) |
| Unknown | 370 | 0.92 (0.88-0.95) | 0.67(0.56-0.77) | 0.86 (0.78-0.94) | 0.79 (0.76-0.82) | 0.33 (0.27-0.39) | 0.98 (0.97-0.99) |

Patient's race

| | | | | | | | |
|---|---|---|---|---|---|---|---|
| White | 2,738 | 0.93 (0.91-0.94) | 0.72(0.68-0.75) | 0.88 (0.85-0.90) | 0.78 (0.77-0.80) | 0.32 (0.30-0.34) | 0.98 (0.98-0.99) |
| Black | 244 | 0.91 (0.87-0.94) | 0.82(0.75-0.89) | 0.88 (0.82-0.94) | 0.73 (0.69-0.78) | 0.49 (0.43-0.57) | 0.95 (0.93-0.98) |
| Asian | 164 | 0.94 (0.89-0.97) | 0.83(0.71-0.93) | 0.92 (0.86-0.98) | 0.71 (0.65-0.76) | 0.44 (0.36-0.52) | 0.97 (0.95-0.99) |
| Other/Unknown | 790 | 0.92 (0.90-0.94) | 0.65(0.57-0.73) | 0.91 (0.86-0.94) | 0.75 (0.73-0.77) | 0.33 (0.29-0.37) | 0.98 (0.98-0.99) |

MRI scanner magnet strength

| | | | | | | | |
|---|---|---|---|---|---|---|---|
| 1.5 T | 2,102 | 0.93 (0.91-0.94) | 0.67(0.62-0.72) | 0.84 (0.80-0.87) | 0.86 (0.85-0.87) | 0.36 (0.32-0.39) | 0.98 (0.98-0.99) |
| 3 T | 1,834 | 0.92 (0.91-0.93) | 0.75(0.72-0.79) | 0.92 (0.90-0.94) | 0.66 (0.65-0.68) | 0.34 (0.32-0.36) | 0.98 (0.97-0.98) |

TABLE 9
AI system's performance on the N test set per exam indication. Indications were automatically
extracted from radiology reports using regular expressions. The extraction was possible for 1,883
(47.8%) examinations. In the remaining 2,053 exams, the script was not able to extract the exam
indication accurately due to the unstructured and narrative reporting. Results for all metrics
are presented with 95% confidence intervals (estimated with bootstrap; N = 2,000 replicates).
To calculate sensitivity and specificity from AI predictions, a decision threshold was selected
such that the overall AI system's sensitivity closely matched average reader sensitivity

| Exam indication | n | AUC ROC | AUC PR | Sensitivity | Specificity |
|---|---|---|---|---|---|
| High-risk screening | 903 | 0.85 (0.75-0.93) | 0.24 (0.07-0.43) | 0.73 (0.54-0.91) | 0.81 (0.79-0.83) |
| Other indication | 980 | 0.93 (0.92-0.94) | 0.84 (0.81-0.88) | 0.91 (0.88-0.93) | 0.73 (0.71-0.75) |
| Extent of disease | 481 | 0.91 (0.89-0.93) | 0.90 (0.86-0.93) | 0.91 (0.88-0.94) | 0.63 (0.58-0.67) |
| Follow-up/surveillance | 286 | 0.91 (0.84-0.97) | 0.02 (0.01-0.07) | 1.00 (1.00-1.00) | 0.78 (0.74-0.81) |
| Further evaluation | 110 | 0.87 (0.76-0.98) | 0.49 (0.05-0.87) | 0.86 (0.50-1.00) | 0.77 (0.71-0.82) |
| Other | 103 | 0.93 (0.84-0.99) | 0.75 (0.49-0.94) | 0.90 (0.73-1.00) | 0.81 (0.75-0.87) |
| Unknown | 2,053 | 0.91 (0.90-0.93) | 0.63 (0.57-0.68) | 0.87 (0.84-0.90) | 0.77 (0.76-0.78) |
| Total | 3,936 | 0.92 (0.92-0.93) | 0.72 (0.69-0.75) | 0.90 (0.79-0.98) | 0.80 (0.73-0.86) |

TABLE 10
Breast-level breakdown of labels in the N data set. Malignant
and benign labels are not mutually exclusive. A patient can have
both a malignant and a benign change in the same breast.

| | Training set | Validation set | Test set | Total |
|---|---|---|---|---|
| Left benign | 2,117 | 518 | 715 | 3,350 |
| Right benign | 2,111 | 477 | 705 | 3,293 |
| Left malignant | 1,278 | 326 | 478 | 2,082 |
| Right malignant | 1,211 | 293 | 427 | 1,931 |
| Left negative | 11,539 | 2,747 | 2,992 | 17,278 |
| Right negative | 11,617 | 2,798 | 3,060 | 17,475 |

TABLE 11
Downstream model performance. Reported values
are n (95% confidence intervals).

| | Gradient Boosting | | Neural Network | |
|---|---|---|---|---|
| | | Accelerated | | Discrete Time |
| | Classifier | Failure Time | Classifier | Model |
| Average C-index | 0.703 +− | 0.672 +− | 0.661 +− | 0.687 +− |
| (average over | 0.001 | 0.004 | 0.002 | 0.002 |
| 10% of top | | | | |
| models from | | | | |
| hyperparameter | | | | |
| search) | | | | |

### Example 1: Exemplary Procedure

The exemplary embodiments of the systems, methods and computer-accessible medium according to the present disclosure can include a subject (e.g., a patient) in need of inspection for possible cancer (e.g., breast cancer) going to a medical clinic to receive a cancer assessment. At the clinic, the subject can receive a breast MRI, a histological sampling of the breast tissue, biopsy of the breast tissue, blood draw, or a combination thereof. Clinical variables, such as medical history can be recorded in the form of electronic health record. Routine histopathological fixation and staining of the breast tissue on a slide(s) can be performed. Assessment of cancer biomarkers can be performed from the blood sample or the breast biopsy. The exemplary embodiments of the systems, methods and computer-accessible medium according to the present disclosure can then incorporate the MRI images, the histopathological slides, or clinical variables to arrive at a medical conclusion (e.g., diagnosis, prognosis, treatment plan).

### Example 2: MRI Feature Extractor Pre-Training and Evaluation

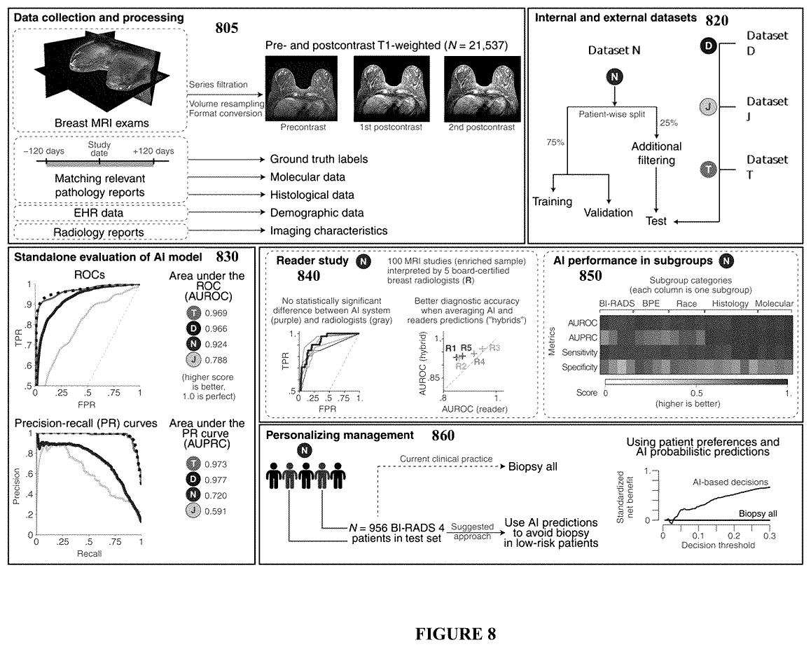

The exemplary feature extractor according to exemplary embodiments of the systems, methods and computer-accessible medium according to the present disclosure, can utilize a deep learning (DL) system such as that illustrated in FIG. 8, that was trained in a supervised manner. The exemplary system shown in FIG. 8 can include, e.g., data collection and processing 805, internal and external datasets 820, standalone evaluation of AI model 830, reader study 840, AI performance in subgroups 850, and personalizing management 860. The feature extractor was provided with many examples of inputs and correct outputs. The inputs of the feature extractor were dynamic contrast-enhanced MRI (DCE-MRI) pre- and postcontrast sequences, all stored as three-dimensional (3D) volumes. This approach mimicked clinical practice, in which radiologists evaluate changes in contrast enhancement in breast to correctly identify suspicious areas. DCE-MRI volumes were passed through the model, which generated predictions of the breast-level probability of malignancy (POM). For each of the patient's breasts, the feature extractor produced a number in a range between 0 and 1. The underlying neural network of the feature extractor performed 3D convolutions, which are mathematical operations that ultimately allow extraction of spatiotemporal features of the inputs.

The dataset used for model training and evaluation included 21,537 bilateral DCE-MRI examinations (n=13,463 patients) who underwent a breast MRI between 2008 and 2020. All examinations were performed with either 1.5-T magnet or 3-T magnet MRI scanners (Table 4). Data included patients reporting for high-risk screening, preoperative planning, routine surveillance, follow-up after suspicious findings in previous MRI examinations, and problem solving (workup of equivocal findings reported in mammography or ultrasound). Patients after bilateral mastectomy, patients after neoadjuvant chemotherapy, and patients with MRI performed to assess implant integrity were excluded. T1-weighted fat-saturated precontrast and at least two postcontrast series were required for the imaging exam to be included in the dataset. The entire dataset was initially split into training, validation, and test subsets with a 60:15:25 ratio. Additional filtering and manual evaluation were performed to ensure consistency of the dataset and ground truth labels. In addition to DCE-MRI examination, associated radiology and pathology reports and patient demographic data (Tables 1 and 2) were collected.

The exemplary feature extractor of exemplary embodiments of the systems, methods and computer-accessible medium according to the present disclosure performance was validated on three international datasets. The first dataset was collected between 2019 and 2021 and contains 394 DCE-MRI examinations. The remaining two datasets contained 922 MRI examinations and 131 examinations from the T dataset. MRI scanner breakdown is described Table 4.

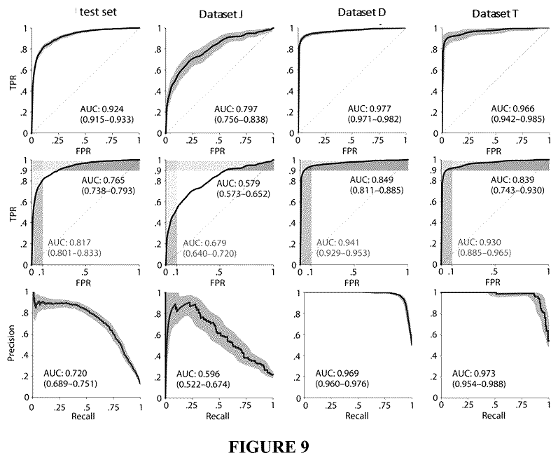

FIG. 9 shows that the feature extractor of exemplary embodiments of the systems, methods and computer-accessible medium according to the present disclosure achieved 0.924 AUROC [95% confidence interval (CI): 0.915 to 0.933] and 0.720 AUPRC (0.689 to 0.751) when classifying breast imaging exams in the internal test set. With respect to FIG. 9, Row 1 illustrates ROC curves with 95% CIs calculated with bootstrapping. Row 2 shows ROC curves with partial AUC (pAUC). The AUCs in Row 2 Bottom represent the pAUC for specificity of 90 to 100%. The AUCs in Row 2 Top represent the pAUC for sensitivity of 90 to 100%. Row 3 shows PRCs with 95% Cis. Partial AUROC (pAUC) at 90% sensitivity was 0.765 (0.738 to 0.793) and 0.817 (0.801 to 0.833) at 90% specificity. Summary of the primary model performance is presented in Table 3.

On the J test set, the feature extractor achieved 0.797 AUROC (0.756 to 0.838) and 0.596 AUPRC (0.522 to 0.674). On the D dataset, the feature extractor reached 0.969 AUROC (0.960 to 0.976) and 0.977 AUPRC (0.971 to 0.982), and on the T dataset, the feature extractor reached 0.966 AUROC (0.942 to 0.985) and 0.973 AUPRC (0.954 to 0.988).

In a retrospective reader procedure, five radiologists interpreted 100 cases sampled from the N test set. All radiologists were board-certified and had between 2 and 12 years of experience interpreting breast MRI exams. Readers achieved an average performance of 0.890 AUROC (range: 0.850 to 0.948) and 0.758 AUPRC (range: 0.712 to 0.868). The feature extractor standalone performance on the reader study subset was 0.924 (0.880 to 0.962) AUROC and 0.784 (0.656 to 0.887) AUPRC. The difference between the feature extractor and radiologists was not statistically significant (Obuchowski-Rockette model, 95% CI AUC difference: 0.09, −0.02; P=0.19). In a head-to-head comparison between the feature extractor's and radiologists' AUROC, the feature extractor was significantly better (P<0.05) than two of the readers (#1 and #2). ROC curves for all readers are presented in FIG. 12, and numerical results are in Table 5. With respect to FIG. 12, the top row shows ROC curves for each of the 5 readers and the bottom row shows PR curves for these readers. All ROC and PR curves are non-parametric (empirical) and were generated from predictions of probabilities of malignancy provided by radiologists. All curves are displayed with 95% confidence intervals estimated with bootstrap (N=2,000 replicates). TPR, true positive rate; FPR, false positive rate.

To understand the difference in the feature extractor's performance on the N test set and the J test set, an additional reader study on the J test set was performed. The additional reader study included two radiologists (also included in the N reader study) who interpreted 97 cases (including 35 cancer cases) sampled from the J test set. The feature extractor achieved 0.802 (0.712 to 0.881) AUROC, whereas the two radiologists achieved 0.787 (0.708 to 0.859) and 0.849 (0.776 to 0.916), respectively. Detailed results are in Table 6.

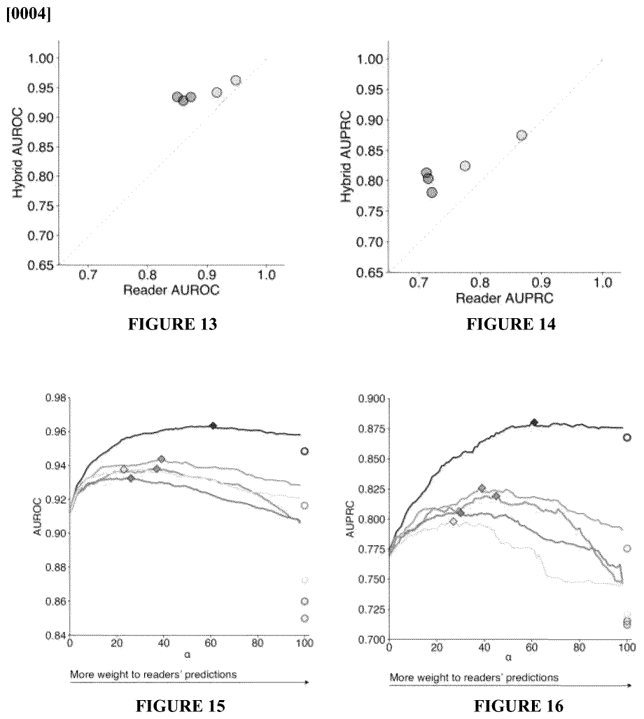

The performance of hybrid models was evaluated by averaging radiologists' and the feature extractor's predictions on the N reader study subset. Hybrid predictions were calculated by averaging predictions of POM made by a radiologist and the feature extractor. Hybrid predictions are simulated, as radiologists were not presented with the feature extractor outputs during exam interpretation. On average, an equally weighted hybrid improved the AUROC by 0.05 and AUPRC by 0.07. This effect was observed each time each radiologist's predictions were averaged, and is illustrated in FIG. 13 showing that an equally weighted average of radiologists and AI model predictions (a hybrid) on the N reader study subset resulted in a stronger performance in terms of AUROC. As shown in FIG. 13, each circle represents the results of each radiologist and the corresponding hybrid. If the circle is above the diagonal, then the hybrid had better results than the reader. Using the feature extractor's predictions, even with a very small weight (for example, 5 to 10% weight), improved the overall outcome.

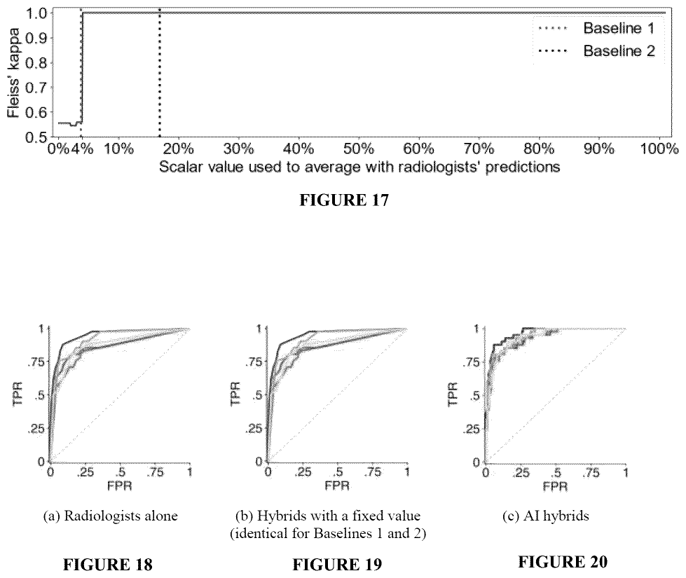

This weight could be treated as an operating point and be set specifically for different readers, as the optimal operating point (that maximizes the performance) varied between radiologists, as evidenced in FIGS. 15 and 16, which show how AUROC (see FIG. 15) and AUPRC (see FIG. 16) change when the a multiplier changes. At α=0%, the hybrid performance is equal to the model only performance. At α=100%, the hybrid performance is equal to the reader only performance (here plotted as an empty circle on the far right of the Figures). Results demonstrated that utilizing AI predictions even at low weights (high α) improved performance. Each line represents performance for a different reader. Diamond-shaped points represent maximum performance for each metric and reader. When analyzing ROC curves for readers and hybrids, as well as Fleiss kappa metrics, the hybrids had higher inter-reader agreement compared to radiologists as illustrated in Table 7 as well as FIGS. 17-20. For example, FIG. 17 illustrates changes in Fleiss' kappa for hybrids with a fixed scalar, depending on the scalar value while FIGS. 18-20 illustrate ROC curves for all readers and hybrids. As averaging with a constant did not change ROC curves, variance of AUROCs within the hybrids did not change for Baselines 1 and 2 (s2=1.7×10−3). Within AI hybrids, variance was lower (s2=2.1 ×10-4) than within radiologists alone.

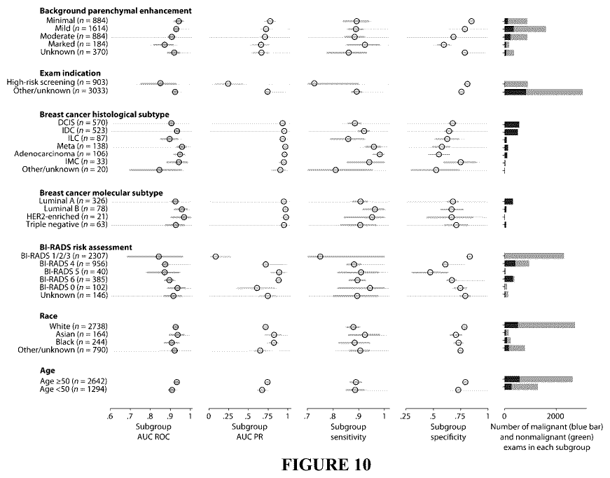

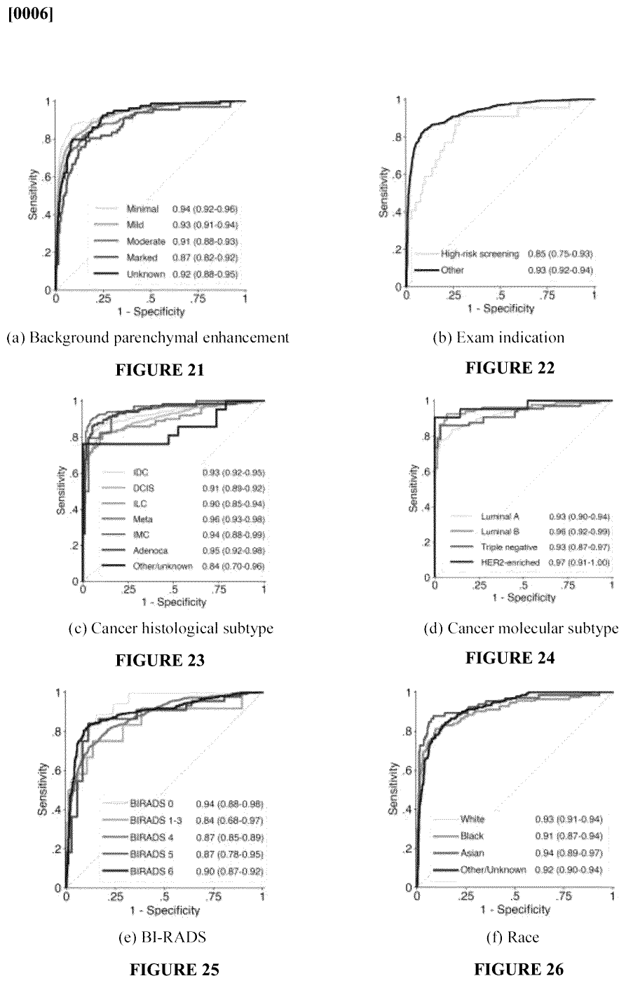

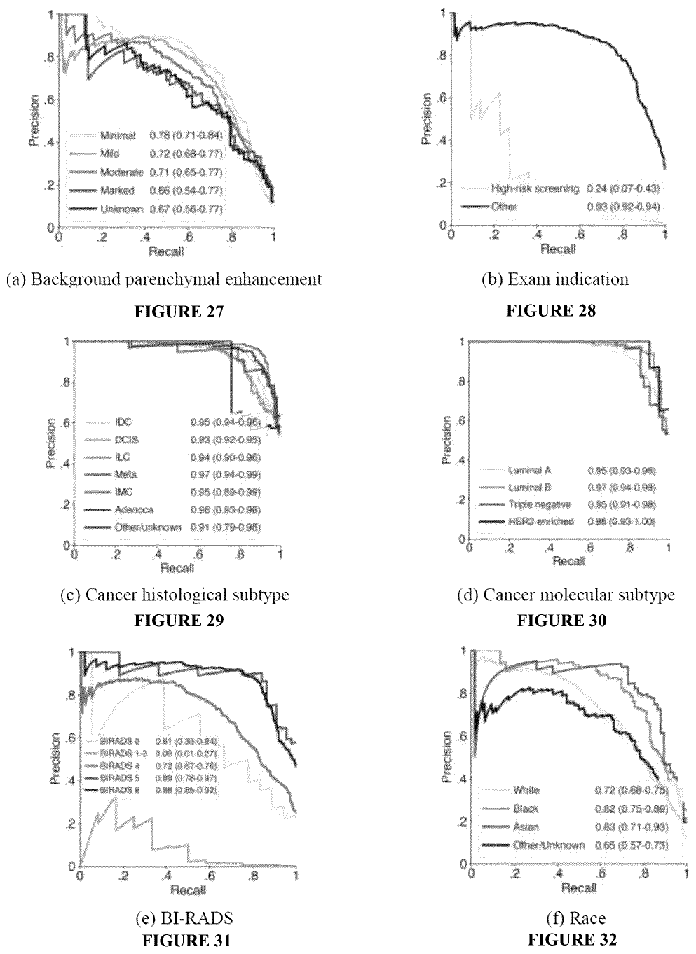

Analyses on a wide range of patient subgroups in the test set were performed, dividing the subgroups with respect to imaging features, cancer subtypes, and other characteristics. For example, FIG. 10 illustrates the feature extractor's performance in key subgroups on the internal test set. Each subgroup was evaluated using four metrics: area under the receiver operating characteristic curve (AUC ROC), area under the precision-recall curve (AUC PR), sensitivity, and specificity. Black (malignant cases) and gray (nonmalignant cases) bars to the right represent the number of examinations in each subgroup. Values for all subgroups and metrics are presented with 95% CIs calculated by bootstrap (N=2000 replicates). To calculate the sensitivity and specificity, a decision threshold was selected such that the feature extractor's sensitivity closely matches the average reader sensitivity. Full numerical values for each subgroup are available in Table 7. Examinations with BI-RADS categories 1, 2, and 3 were aggregated because there were no MRI exams associated with malignant diagnoses in BI-RADS 1 and 2 categories; thus, AUROC would be undefined in those subgroups. HER2, human epidermal growth factor receptor 2. DCIS, ductal carcinoma in situ; IDC, invasive ductal carcinoma; ILC, invasive lobular carcinoma; IMC, invasive mammary carcinoma. The analyses were performed to establish whether the model is underperforming in any of the evaluated categories and to investigate whether some groups are more likely to benefit more from the model. Full numerical values of the subgroup analysis are in Tables 8 and 9, and select ROC/precision-recall (PR) curves are illustrated in FIGS. 21-32. In FIGS. 21-32, as no malignant examples existed in BI-RADS 1 and 2 categories in the test set, AUC ROC and AUC PR would not be defined for those groups. BI-RADS 1 and 2 were combined with BI-RADS 3 to generate curves and calculate AUC ROC and AUC PR.

The performance in subgroups with respect to exam indications were compared and no statistically significant differences (P≥0.05) were found. For example, no difference was observed in model performance in women undergoing high-risk screening MRI versus women undergoing a follow-up exam (P=0.4). No difference was observed when comparing performance in screening MRI versus the extent of disease MRI (P=0.22) or screening MRI versus any nonscreening MRI exam (P=0.11). Model performance in all exam indication subgroups is described in Table 8.

No differences were observed in the feature extractor's performance between patients with various histological cancer subtypes, even when comparing more common cancers (for example, invasive ductal carcinoma) with less common malignancies (for example, invasive lobular carcinoma; AAUC: 1.5; two-sided DeLong's test, P=0.15). When considering patient demographics, the results indicated that the feature extractor appeared to be unbiased, even when the subgroup was not as commonly represented in the training set, as in the case of Black women [n=802 patients in the training set; test set AUROC of 0.91 (0.87 to 0.95)] versus white women [n=9819 in the training set; test set AUROC of 0.93 (0.91 to 0.94)]. The AUC difference between those two groups was not statistically significant (P=0.33).

### Example 3: Exemplary Mri Feature Extractor Diagnosis Testing

Personalized Management of Patients with BI-RADS 3 and BI-RADS 4 Lesions

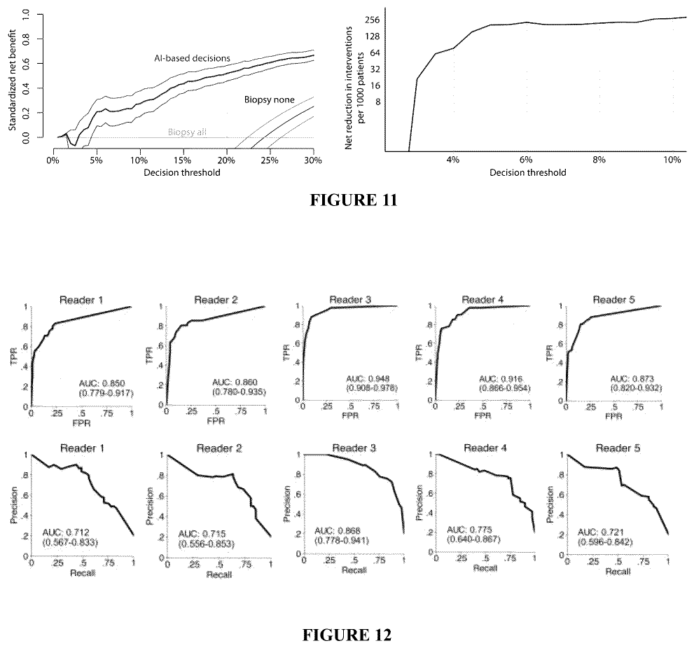

To create a diagnostic decision-making application of the feature extractor of exemplary embodiments of the systems, methods and computer-accessible medium according to the present disclosure, using the feature extractor predictions as an aid in downgrading BI-RADS 4 lesions to BI-RADS 3 was used as a test. Definitions of BI-RADS risk assessment categories were used as specified in the American College of Radiology BI-RADS Atlas fifth edition. The analysis was conducted in two ways, both using the full test set and original BI-RADS of the imaging exams as reported initially by radiologists. First, trade-offs between correctly avoided biopsies and missed cancers at various decision thresholds used to binarize probabilities of malignancy was compared directly. The trade-off was an equally weighted comparison between the number of successfully opted out patients (patients who avoided unnecessary biopsy) and missed cancers. In a second approach, the decision curve analysis (DCA) methodology was used to incorporate patients' and clinicians' preferences into decision-making and explored whether using the model could be clinically beneficial or harmful. FIG. 11 illustrates exemplary results of the DCA support using the feature extractor for making diagnostic decisions in low-risk patients with BI-RADS 4 lesions.

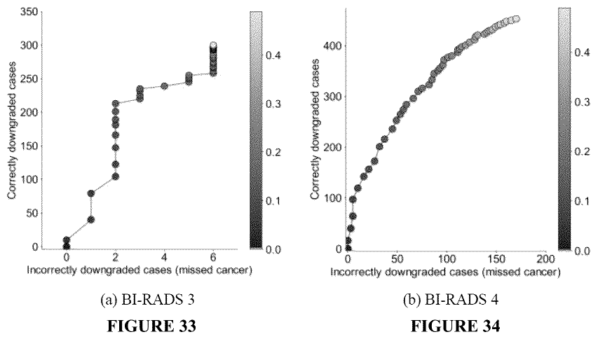

According to exemplary embodiments of the systems, methods and computer-accessible medium according to the present disclosure, at an operating point that allows for the identification of 5.4% nonmalignant BI-RADS 4 lesions (avoiding a biopsy), no cancers would be missed. At a different operating point at which the system correctly determined 22.9% of BI-RADS 4 lesions to be nonmalignant (avoiding a biopsy), 10 (2.3%) malignant BI-RADS 4 findings would be missed (determined as nonmalignant by the system). For example, FIGS. 33 and 34 illustrate the trade-off in missed cancers versus correctly avoided biopsies when using only the feature extractor to decide on management.

Similarly, BI-RADS 3 lesions could potentially be downgraded to BI-RADS 2, subsequently leading to patients' return to routine screening instead of short-term follow-up MRI after 6 or 12 months. The feature extractor of exemplary embodiments of the systems, methods and computer-accessible medium according to the present disclosure correctly downgraded 235 BI-RADS 3 lesions (73.2% of all nonmalignant BI-RADS 3 cases) to BI-RADS 2, missing three cancer cases.

The use of the feature extractor in patients with BI-RADS 4 lesions resulted in net reduction in interventions in low decision thresholds. If a decision threshold is at 5%, the approach resulted in a net reduction of 156 breast biopsies per 1000 patients.

### Exemplary Reader Study Error Analysis

An error analysis of the feature extractor's predictions using the N reader study subset was performed. Predictions of malignancy made by the feature extractor of exemplary embodiments of the systems, methods and computer-accessible medium according to the present disclosure were compared to predictions made by radiologists. Assessment indicated the feature extractor's predictions matched those of the radiologists. FIG. 33 shows the trade-off when using only feature extractor's predictions to decide whether the patient should return for a 6-month follow-up or not in cases with BI-RADS 3 findings. Correctly downgraded patients would return to a regular screening, while missed cancers prevent the opportunity to detect cancer if imaging would be performed again in 6 months. FIG. 34 shows the trade-off in BI-RADS 4 cases. Correctly downgraded cases from BI-RADS 4 to BI-RADS 3 represent patients who would avoid an unnecessary biopsy (by downgrading BI-RADS 4 lesion to BI-RADS 3), while missed cancers are situations wherein patients do have breast cancer but would not be subject to biopsy due to the feature extractor's predictions. Both FIGS. 33 and 34 do not take into consideration patient's and physician's preferences and do not weigh the trade-off items (e.g., one missed cancer case is more important than one avoided biopsy). FIGS. 33 and 34 also ignore the potential effect of a physician ultimately making a decision based on their own knowledge supported by the feature extractor. FIGS. 33 and 34 show exemplary illustrations of the trade-off at different operating points. Operating points are color-coded by increasing binarization thresholds (lighter colors are higher thresholds).

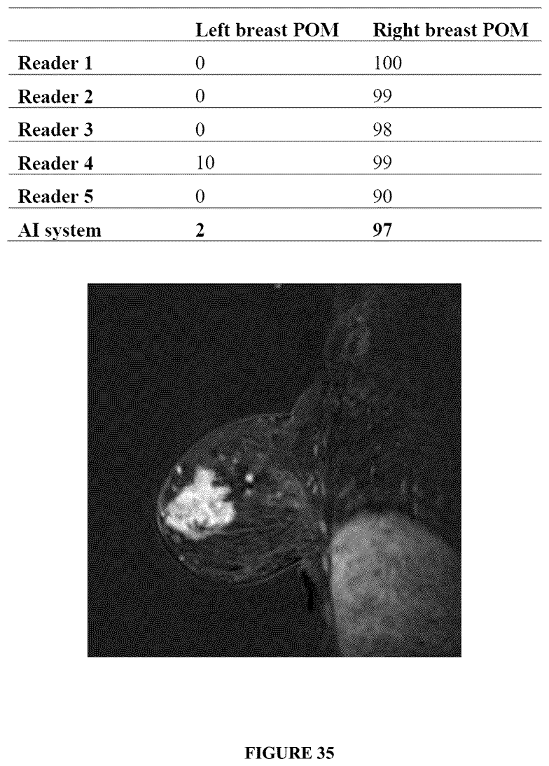

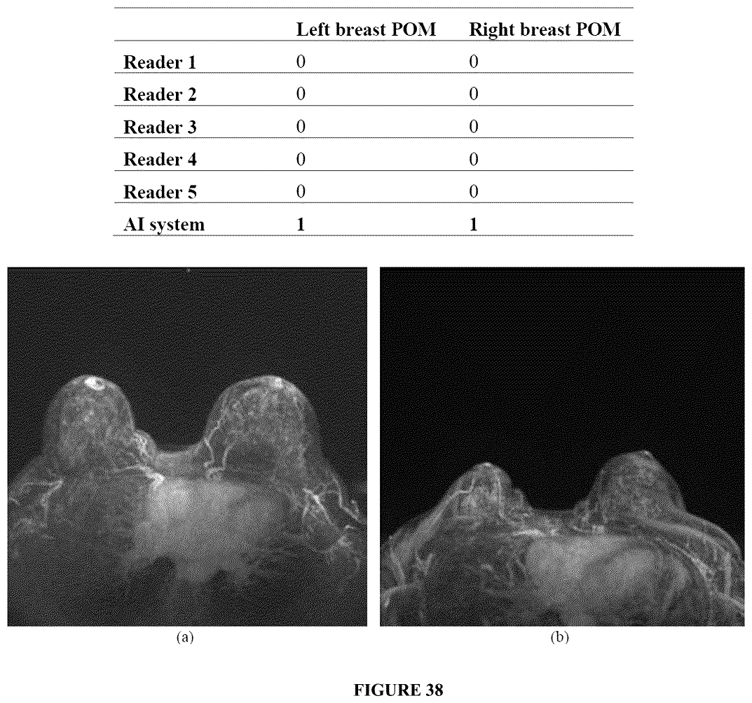

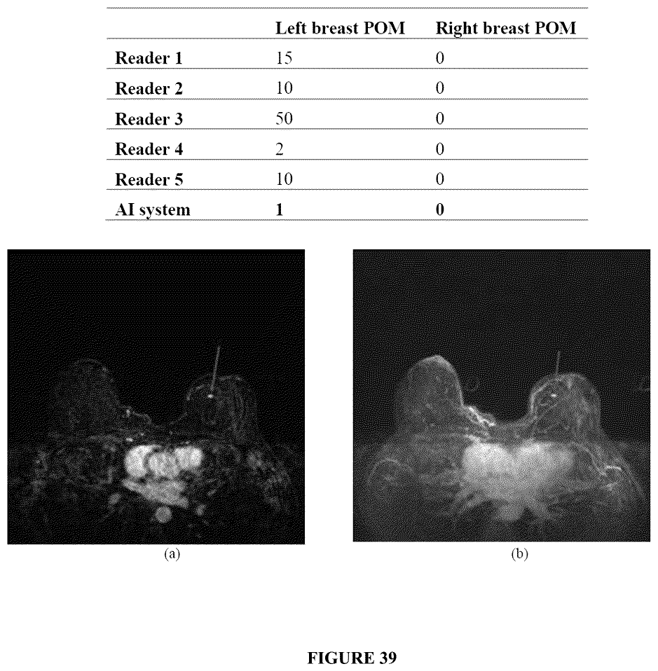

The feature extractor of exemplary embodiments of the systems, methods and computer-accessible medium according to the present disclosure behaved correctly when predicting examinations with cancer by giving them a high POM (e.g., see FIGS. 35-37) and when correctly identifying negative examinations by giving a very low probability (e.g., FIG. 38). In some benign cases, the feature extractor's predictions were lower than were those of radiologists, demonstrating potential to avoid unnecessary biopsies (e.g., FIG. 39).

FIG. 35 shows an exemplary illustration of a case study where all five radiologists assessed a very high probability of malignancy in the right breast (one BI-RADS 4C, four BI-RADS 5). The feature extractor of exemplary embodiments of the systems, methods and computer-accessible medium according to the present disclosure also correctly identified the malignancy and gave the examination a 97% probability of cancer in the right breast. One radiologist found a suspicious lesion in the left breast. Based on the patient's history, that lesion was also identified by the radiologist originally interpreting the exam. Upon biopsy, the lesion was found to be benign.

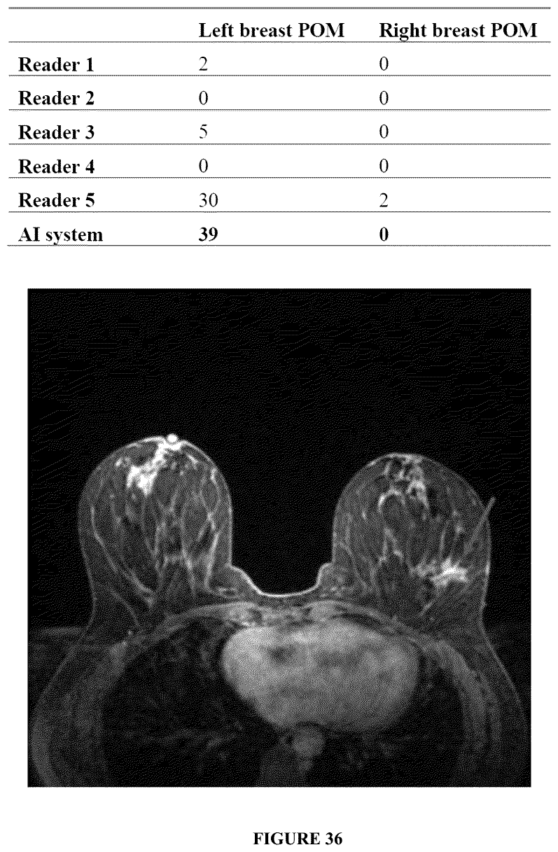

FIG. 36 shows an exemplary illustration of a case study where three out of five readers found lesions in the examination. Out of the three who did, only one assessed a high probability of malignancy (reader 5, 30%). The suspicious lesion was later confirmed to be malignant. The feature extractor of exemplary embodiments of the systems, methods and computer-accessible medium according to the present disclosure correctly predicted the malignancy, giving a 39% probability in the left breast, and 0% POM in the right breast.

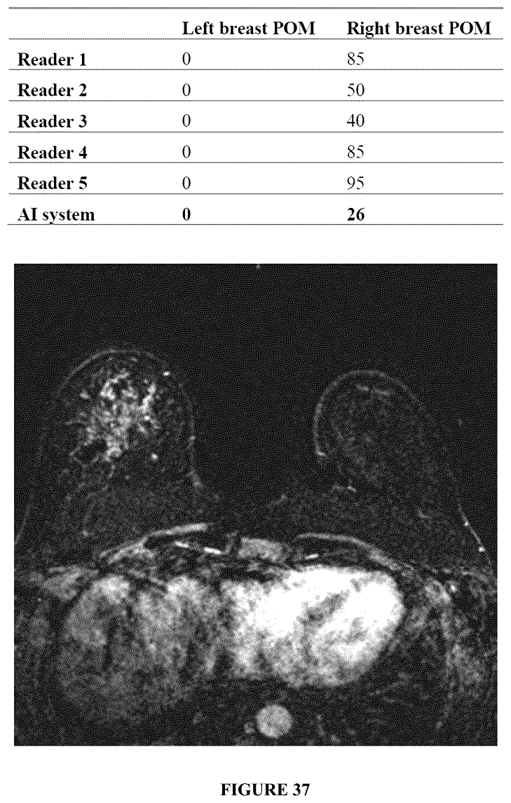

FIG. 37 shows an exemplary illustration of a case study where the examination was performed in the diagnostic process of evaluating bloody left nipple discharge, which demonstrated atypical cells. Although no suspicious findings occurred in the left breast, all radiologists agreed that the enhancement in the right breast was highly suspicious. This prediction was matched by the feature extractor output according to exemplary embodiments of the systems, methods and computer-accessible medium according to the present disclosure. The lesion was found to be malignant.

FIG. 38 shows an exemplary illustration of two case studies where in two sample imaging exams where all radiologists agreed that no suspicious lesions existed in the exam, and the feature extractor of exemplary embodiments of the systems, methods and computer-accessible medium according to the present disclosure gave very low probabilities of malignancy.

FIG. 39 shows an exemplary illustration of a case study where in an examination where all radiologists would perform a biopsy on the lesion in the left breast. One reader classified this exam as BI-RADS 4A, three readers as BI-RADS 4B, and one as BIRADS 4C. According to patient history, the suspicious lesion in the left breast was studies by biopsy and yielded a benign result. The feature extractor of exemplary embodiments of the systems, methods and computer-accessible medium according to the present disclosure correctly outputted a low POM.

### Example 4: Downstream Task Training and Evaluation

Using the exemplary MRI Data Representations generated by the MRI feature extractor, a Downstream Model to predict 3-year overall breast cancer recurrence was trained according to exemplary embodiments of the systems, methods and computer-accessible medium according to the present disclosure. The Downstream Model was a gradient boosting classifier. The Downstream Model of exemplary embodiments of the systems, methods and computer-accessible medium according to the present disclosure consistently yielded an AUCROC (area under the receiver operating characteristic curve) above 0.7. Selected specific patient subgroups of clinical interest was evaluated with the Downstream Model. For example, a subgroup of hormone-positive, HER2-negative patients, who also received the Oncotype DX recurrence prognostic test (based on genomic data) was evaluated. On the N dataset of patients with available Oncotype DX scores (n≈125), the Downstream Model of exemplary embodiments of the systems, methods and computer-accessible medium according to the present disclosure resulted in a 0.186 (from 0.537 to 0.723) improvement in AUCROC. In the group of patients with available Oncotype DX scores who received adjuvant chemotherapy, the Downstream Model had a 0.123 AUCROC improvement (from 0.597 to 0.720) over Oncotype DX. In the group of patients with available Oncotype DX scores who did not receive adjuvant chemotherapy (n=81), the Downstream Model had a 0.202 AUCROC improvement (from 0.535 to 0.737) over Oncotype DX.

Various model architectures and statistical approaches to predicting breast cancer recurrence were tested according to exemplary embodiments of the systems, methods and computer-accessible medium according to the present disclosure. Two types of model architectures were tested: a gradient boosting model and neural network. These models were trained with various statistical approaches to predicting cancer recurrence (classifier, accelerated failure time model, discrete time model). Clinical Variables were used as input. Results are shown in Table 11.

### Example 5: Exemplary Methods

### Demographic Data and Imaging Characteristics

Table 1 summarizes the demographic data and imaging characteristics in the datasets. Values are n (%) unless specified otherwise. BI-RADS risk assessment categories, background parenchymal enhancement (BPE), and the amount of fibroglandular tissue are reported according to the American College of Radiology BI-RADS Atlas fifth edition. Breast-level diagnosis statistics are presented in Table 10. Malignant and benign findings are not mutually exclusive. Thus, the total number of examinations labeled as malignant, benign, or negative can be greater than 100%. A negative diagnosis means that no pathology reports were associated with an examination.

### Exemplary N Health Dataset

The dataset consists of 21,537 DCE-MRI examinations from 13,463 patients who underwent DCE-MRI between 2008 and 2020. The data were randomly split into training, validation, and test sets with 60, 15, and 25% of the data, respectively. This split was made on a patient level, so that data from one patient could be in only one subset.

### Exemplary Ground Truth

All datasets used to train and evaluate the feature extractor of exemplary embodiments of the systems, methods and computer-accessible medium according to the present disclosure contain breast-level labels describing the presence or absence of benign or malignant findings in either the left or right breast. All benign and malignant labels in the dataset were pathology-proven based on specimen analysis from either a breast biopsy or surgery, done by searching and matching all pathology reports dated 120 days before or after the day of examination.

### Exemplary Filtering the Dataset

To maximize the accuracy of the truthing and to remove potentially confounding subgroups, additional filtering of the dataset was performed. In the full dataset, imaging exams where a cancer label was not reliably determined or technical issues with data extraction or data consistency were excluded. A set of rules specific to the test set to further remove label noise was added. Patients with a history of bilateral mastectomy (n=165), patients after neoadjuvant chemotherapy (n=105), and patients with breast implants (n=370) were excluded. Cases were reviewed manually where (i) an examination was initially labeled by the feature extractor as negative and, at the same time, was assigned as BI-RADS 1, 2, or 3 by a radiologist; (ii) an examination was labeled as malignant but was assigned as BI-RADS 1 or 2; and (iii) an examination was labeled as malignant but was assigned as BI-RADS 0, 3, or 6. In situation (i), a 1-year negative follow-up requirement was added, meaning that in the year after the MRI exam date, (a) no pathology reports were associated with the patient; (b) at least one breast imaging exam (mammography, MRI) occurred with BI-RADS category 1, 2, or 3; and (c) no breast imaging exams had BI-RADS category 0, 4, 5, or 6. Negative studies without proper follow-up (n=1135) were excluded. In situation (ii), cases (n=10) with clear mistake to label an examination as both malignant and BI-RADS 1 or 2 were excluded. In situation (iii), all cases (n=433) were manually reviewed and verified correctness of the labels. If necessary, examinations had their labels fixed or were excluded (n=19).

### Exemplary External Datasets

Datasets were collected. All external data underwent the same preprocessing pipeline. The sets were resampled, reoriented to the LPS (left-posterior-superior) orientation, and saved in an appropriate file format.

### Exemplary J Dataset

This exemplary dataset includes 394 DCE-MRI examinations. In 145 imaging exams, at least one breast had pathology-proven breast cancer. In the remaining 249 examinations, no pathology-confirmed cancer was found in any breast.

Labeling and anonymization were performed by a board-certified breast radiologist, and the labels were pathology-proven. Ninety-nine percent of examinations were acquired on a 1.5T Siemens MAGNETOM Sola between December 2019 and August 2021. Indications for a scan varied, but the largest group was patients undergoing a problem-solving MRI after ambiguous findings in other modalities.

After obtaining the dataset, all imaging exams were semi-automatically identified pre- and postcontrast sequences and converted to the NIfTI format. Then, a manual visual review of saved images was performed to confirm accuracy of pre-/postcontrast assignment. From the original dataset, five examinations that did not contain fat-saturated images, three unilateral exams, two examinations missing pathology confirmation, one exam that was confirmed to be postneoadjuvant chemotherapy MRI, and one examination that did not have consistent image size in pre- and postcontrast series were excluded.

### Exemplary D Dataset

The dataset contained 922 examinations (n=922 patients) with invasive breast cancer, meaning that for each of these exams, at least one breast had pathology-proven breast cancer. The dataset was accompanied by detection labels, clinical features, and imaging features. Detection labels were available for all imaging exams and were shared in a tabular form describing the coordinates of 3D cuboid bounding boxes. Images in the D dataset were stored in DICOM format and were preprocessed with the same pipeline as the N dataset. Images were resampled and reoriented to the LPS orientation. Pre- and post-contrast sequences, as required by our model, and performed inference to generate predictions were identified.

Bounding box labels were converted to breast-level labels based on the middle point of bounding boxes. For example, if the central point of a bounding box was located on the left anatomical side of the patient, then a label for left breast was generated. The images were annotated by eight fellowship-trained breast radiologists with 1 to 22 years of post-fellowship experience.

### Exemplary T Dataset

Originally, this dataset included 164 examinations from 139 patients and contains images, clinical data, biomedical data, and DICOM Structured Reporting files. The data in its original form were not suitable for the feature extractor's evaluation. Therefore, a pipeline for generating feature extractor-ready T data with labels was developed. After processing, the dataset contained 131 imaging exams.

### The Feature Extractor

### Exemplary Inputs and Outputs

Let x∈ [symbol] C, Z, X, Y denote an input. Z, X, and Y are the spatial dimensions of an MRI volume, and C channels are different MRI sequences, that is, pre- and postcontrast series. The neural network of exemplary embodiments of the systems, methods and computer-accessible medium according to the present disclosure can generate four probability estimates y\hat{ }_(lb,) y\hat{ }_(lm,) y\hat{ }_(rb,) y\hat{ }_(rm,)∈[0,1] that indicate the predicted probability of the presence of benign and malignant lesions in each of the patient's breast (b and m represent benign and malignant findings, and l and r represent left and right breasts, respectively). Probabilities of benign findings (y\hat{ }_(lb,) y\hat{ }_rb) can be used only as a multitask learning regularization method. Predicted probabilities of malignant findings (y\hat{ }_(lm,) y\hat{ }_rm) may be evaluated.

### Exemplary Architecture

The feature extractors of exemplary embodiments of the systems, methods and computer-accessible medium according to the present disclosure can be deep residual neural networks with 3D convolutions to detect spatiotemporal features. 3D-ResNet18 backbone can be used with a max pooling layer before linear classifier.

Feature extractors according to the exemplary embodiments of the systems, methods and computer-accessible medium of the present disclosure with pretrained backbones can perform better compared to models trained from scratch. Weights can be used from models pretrained on the Kinetics-400 dataset, which is an action recognition in video dataset.

### Exemplary Data Augmentation During Training

Various geometrical, intensity-based and MRI-specific augmentations were tested. Affine transformation resulted in consistently beneficial results. During training of the feature extractor of exemplary embodiments of the systems, methods and computer-accessible medium according to the present disclosure, images can be randomly (P=0.5) flipped horizontally in the left-right anatomical axis. When the volume is flipped, labels are flipped as well, such that y_lb→y_rb while y_rb→y_lb and y_lm→y_rm while y_rm→y_lm.

The affine augmentations applied were not based on the tensor sizes but rather on real-life dimensions. To convert into anatomical dimensions, affine matrices can be calculated for each study that defined relationships between pixel and real-life sizes. To compute an affine matrix, image spacing, origin, and direction cosine values that were collected from DICOM metadata can be used. The matrices were necessary to resample initially to the same pixel spacing and reorient all images to the LPS orientation, which is a standard orientation in DICOM convention. Pixels in the matrix followed the anatomical order and went from right toward left, from anterior toward posterior, and from inferior toward superior. Resampling can be performed using linear interpolation.

### Exemplary Subtraction Images

Subtraction images can be used to improve performance. Subtraction images can be generated by a simple matrix subtraction between postcontrast and precontrast volumes, such that X_(substraction i)=X_(post i)−X_pre, where X_(post) is one of i postcontrast volumes and X_pre is the precontrast volume. Lower range values were not clipped to zero.

### Exemplary Training Details

Most or all feature extractor models according to the exemplary embodiments of the systems, methods and computer-accessible medium of the present disclosure were trained with the Adam optimizer, and top models for the ensemble were selected after hyperparameter tuning with random search. AUROC for malignant labels was the target metric in the hyperparameter search. The following exemplary parameters were tuned:
(1) Scaling ∈[5%, 25%], symmetrically along each axis;
(2) Rotation ∈[5°,30°], symmetrically along each axis;
(3) Translation ∈[5 mm, 20 mm], symmetrically along each axis;
(4) Dropout, 25% chance on the fully connected layer (yes or no);
(5) Label smoothing (39, 40) α∈[0, 0.1];
(6) Stochastic depth rate (41) ∈[0, 0.1];
(7) Weight decay ∈[1e-6, 1e-4];
(8) Learning rate ∈[7e-7, 2e-5];
(9) Number of warm-up epochs ∈[2, 6]; and
(10) Choice of the learning rate scheduler (10× reduction after α epochs versus cosine annealing) and scheduler policies.

Exemplary models according to exemplary embodiments of the systems, methods and computer-accessible medium according to the present disclosure can be trained with mixed precision using NVIDIA Apex open-source library. Network architecture included group normalization. Group normalization with 16 groups can perform well. Neptune.ai and Weights & Biases can be used for tracking, evaluating, and visualizing experimental results.

### Exemplary Test Time Augmentations

During test time, all data samples were transformed 10 times (TTA) and averaged inference results from all TTA samples. This approach according to exemplary embodiments of the systems, methods and computer-accessible medium according to the present disclosure can improve accuracy and robustness of feature extractor models. The optimal TTA policy according to exemplary embodiments of the systems, methods and computer-accessible medium according to the present disclosure was found on a validation set after running a random search for the following TTA hyperparameters: number of TTA rounds ∈[1, 10], affine scaling ∈[10%, 20%], rotation ∈[10°, 20°], and translation ∈[10 mm, 20 mm] using gamma transformations or blurring. The best-performing TTA policy on the validation set was the one that implemented 10 rounds of TTA, random horizontal flips, and affine transformations of 10% scaling factor, 10° rotation, and 10-pixel translation. Differences between various TTA policies were usually indistinguishable. For single models (e.g., not full ensemble), an improvement of ≈0.005 AUCROC and ≈0.01 AUCPR on the validation set can be observed.

### Exemplary Reader Study on the N Subset

A retrospective reader analysis was designed to compare the standalone clinical performance of the model according to exemplary embodiments of the systems, methods and computer-accessible medium according to the present disclosure with radiologists. A single-arm design (readers interpreting MRI examinations only) was included in the study. Five board-certified breast radiology attendings were recruited to participate in the study. 100 imaging exams were randomly selected from the primary test set as a reader study set for readers to interpret. The dataset was enriched with malignant and nonmalignant biopsied cases. Specifically, 40 malignant, 40 benign, and 20 negative studies existed in the reader study set. Readers were informed that the population of the study does not represent typical distribution of patients undergoing breast MRI. Readers had no knowledge about the specific split. Readers were also blinded to any confidential information, prior imaging exams, or indications for the examination. Radiologists had access to all available MRI sequences and were not limited to T1-weighted fat-saturated series that were used as inputs for the feature extractor system.

Readers were provided with a workstation preloaded with examinations. All imaging exams used in the reader study were pseudonymized and stored in a server separate from clinical PACS (picture archiving and communication system) servers. Radiologists had access to the workstation and a data collection tool. Before joining the study, recruited readers had to become familiar with study instructions and the viewer used in the study. Radiologists had to provide the following predictions:
(1) A POM for the whole examination, in a range between 0 and 100.
(2) A POM for the left breast, in a range between 0 and 100.
(3) A POM for the right breast, in a range between 0 and 100.
(4) A forced BI-RADS for the imaging exam. Radiologists could only use BI-RADS categories 1, 2, 3, 4A, 4B, 4C, or 5. BI-RADS category 0 or 6 was not available. If the examination was classified as BI-RADS 4, then radiologists had to specify a subcategory (4A, 4B, or 4C).

Readers were advised to use BI-RADS likelihood of cancer ranges as a guideline when assigning POM values. If a reader believed that the examination was probably benign (BI-RADS 3), then the reader was to assign a POM value in the (0, 2] interval.

### Exemplary Reader Study on the J Subset

To evaluate whether the shift in data distribution across data sets affected the feature extractor's performance to a similar degree as the shift affected radiologists, an additional, smaller reader exemplary analysis was performed. This study was designed in the same manner as the original reader exemplary analysis on the N dataset. The exemplary analysis included two attending breast radiologists, who also participated in the N reader study. The J reader study had a slightly different study enrichment. The J dataset did not distinguish between benign and negative (nonbiopsied) exams. 35 malignant exams and 62 nonmalignant examinations existed in the reader exemplary analysis subset.

### Exemplary Statistical Analysis

To measure model performance according to exemplary embodiments of the systems, methods and computer-accessible medium according to the present disclosure, ROC and PR were used and AUC was calculated for both (AUCROC and AUCPR) using a nonparametric (trapezoidal) method. Sensitivity and specificity were reported. Specific clinical scenarios were evaluated using partial AUC statistic, as implemented in the partial ROC R package. All or most results, where appropriate, were reported with 95% Cis derived using bootstrapping. When evaluating standalone performance versus reader performance, a single-treatment random-reader random-case model was used based on the Obuchowski-Rockette model. This exemplary method accounted for variability both between readers and cases. The null hypothesis for significance testing was that the average breast-level AUC of the feature extractor DL model equaled the average AUC of radiologists. In subgroup analyses, for comparisons of AUCs of two curves, a two-sided DeLong's test was performed. P<0.05 was considered statistically significant.

For DCA, R packages rmda and dcurves were used to generate the curves, calculate net benefit, and avoid net interventions. A range of reasonable threshold probabilities for evaluated subgroups (BI-RADS 4 and 3) was established and a wide range of threshold probabilities on a full population was reported.

The standardized net benefit (sNB; also known as relative utility) for the opt-out policy (here, downgrading BI-RADS 4 to BI-RADS 3) can be defined as

$$\\ sNA ( α ) = TNR ( α ) - ( \frac{prevalence}{1 - prevalence} ) ( \frac{1 - α}{α} ) FNR ( α )$$

where prevalence is disease prevalence, a is the decision threshold, TNR is a true-negative rate, and FNR is a false-negative rate.

To avoid overestimating the net benefit, the results were boostrapped with N=2000 replicates and reported decision curves with 95% CIs.

For measuring interreader variability, Fleiss' kappa was used, specifically with Randolph's free-marginal modification, for agreement between positive and negative cases. Fleiss' kappa was calculated on exam level and breast level for readers. In addition, ROC curves were generated for readers and hybrids and measured the sample variance in AUC ROC. Details can be found in Table 7 and FIGS. 17-20.

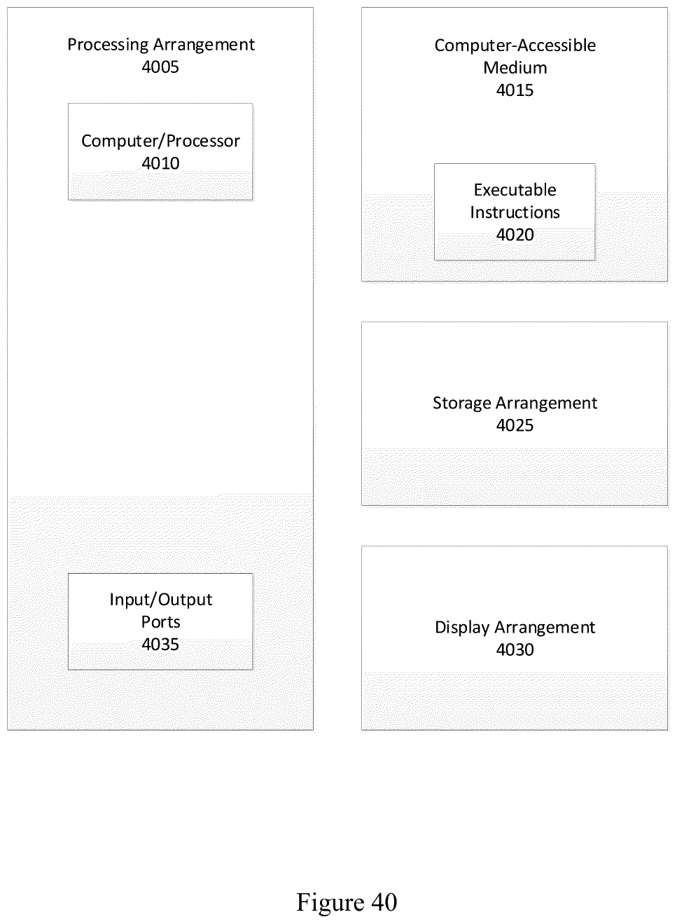

FIG. 40 shows a block diagram of an exemplary embodiment of a system according to the present disclosure, which can be utilized either in part or completely with any one or more of the exemplary embodiments of the present disclosure as provided in the enclosed Appendix. For example, exemplary procedures in accordance with the present disclosure described herein can be performed by a processing arrangement and/or a computing arrangement 4002. Such processing/computing arrangement 4002 can be, for example entirely or a part of, or include, but not limited to, a computer/processor 4004 that can include, for example one or more microprocessors, and use instructions stored on a computer-accessible medium (e.g., RAM, ROM, hard drive, or other storage device).

As shown in FIG. 40, for example a computer-accessible medium 4006 (e.g., as described herein above, a storage device such as a hard disk, floppy disk, memory stick, CD-ROM, RAM, ROM, etc., or a collection thereof) can be provided (e.g., in communication with the processing arrangement 4002). The computer-accessible medium 4006 can contain executable instructions 4008 thereon. In addition, or alternatively, a storage arrangement 4010 can be provided separately from the computer-accessible medium 4006, which can provide the instructions to the processing arrangement 4002 so as to configure the processing arrangement to execute certain exemplary procedures, processes and methods, as described herein above, for example.

Further, the exemplary processing arrangement 4002 can be provided with or include an input/output arrangement 4014, which can include, for example a wired network, a wireless network, the internet, an intranet, a data collection probe, a sensor, etc. FIG. 40 shows that the exemplary processing arrangement 4002 can be in communication with an exemplary display arrangement 4012, which, according to certain exemplary embodiments of the present disclosure, can be a touch-screen configured for inputting information to the processing arrangement in addition to outputting information from the processing arrangement, for example. Further, the exemplary display 4012 and/or a storage arrangement 4010 can be used to display and/or store data in a user-accessible format and/or user-readable format.

According to certain exemplary embodiments of the present disclosure, a method can be provided that comprises:
extracting at least one image feature from a medical image of a tissue to provide an extracted image feature;
extracting at least one histopathology feature from digitized histopathology data of the tissue to provide an extracted histopathology feature;
processing the at least one extracted image feature and the at least one extracted histopathology feature to provide a multi-modal representation of the tissue; and
processing the multimodal representation of the tissue with a specific procedure to provide a prediction regarding a medical outcome of the tissue.

The method of para. [00218] can be provided, wherein the processing the extracted image feature and the at least one extracted histopathology feature to provide a multi-modal representation of the tissue processes the extracted image feature and the extracted histopathology feature with information from an electronic health record corresponding to the tissue to provide the multi-modal representation of the tissue.

The method of para. [00219] can be provided, wherein the information from the electronic health record is at least one of (i) a tumor stage of the tissue, or (ii) a status of a molecular biomarker in a subject that provided the tissue.

The method of para. [00219] can be provided, wherein the specific procedure is at least one of (i) a machine-learning procedure, (ii) a trained, machine-learning procedure, or (iii) a machine-learning procedure trained on a set of magnetic resonance images of one or more cancerous breasts.

The method of para. [00219] can be provided, wherein the medical image is at least one of a magnetic resonance image of a human breast or a human body part that is (i) targeted for an inspection for a possible disease, a cancer or a breast cancer, or (ii) afflicted with a possible disease, a cancer or a breast cancer.

The method of para. [00219] can be provided, wherein the extracted image feature is a visual detail that suggests a presence of a cancer.

The method of para. [00219] can be provided, wherein the digitized histopathology data of the tissue is an image of a digitized histopathology slide of at least one of (i) the tissue, (ii) a human breast, (iii) a human body part that is targeted for an inspection for a possible disease, a possible cancer or a possible breast cancer, or that is afflicted with a breast cancer.

The method of para. [00219] can be provided, wherein the extracted histopathology feature is a visual detail that suggests a presence of a cancer.

The method of para. [00219] can be provided, wherein the tissue is a human breast tissue.

The method of para. [00219] can be provided, wherein the tissue is a human body part that is targeted for an inspection for at least one of (i) a possible disease, or (ii) a possible breast cancer.

The method of para. [00219] can be provided, wherein the tissue is a human breast that is afflicted with a breast cancer.

The method of para. [00219] can be provided, wherein the prediction regarding the medical outcome of the tissue is a likelihood of (i) a relapse of a disease or breast cancer, (ii) a favorable response to a therapy for a condition, cancer, or breast cancer present in the tissue.

The method of para. [00219] can be provided, further comprising administering to a subject that provided the sample a therapeutic intervention that corresponds to the prediction regarding the medical outcome of the tissue.

The method of para. [00219] can be provided, wherein the tissue is of a subject, further comprising administering to the subject a therapeutically-effective amount of a therapeutic intervention based at least in part on the prediction regarding the medical outcome of the tissue.

According to another exemplary embodiment of the present disclosure, a method can be provided comprising analyzing on a computer system a feature of a medical image of a tissue and a visual feature of a histopathology sample of the tissue using a specific procedure, wherein the specific procedure is a machine-learning procedure trained on a set of images of cancerous anatomies.

The method of para. [00232] can be provided, wherein the analyzing is of the feature of the medical image of the tissue, the visual feature of the histology sample, and information from an electronic health record corresponding to the tissue.

The method of para. [00232] can be provided, wherein the information from the electronic health record is at least one of (i) a tumor stage of the tissue, or (ii) a status of a molecular biomarker in a subject that provided the tissue.

The method of para. [00232] can be provided, wherein the images of cancerous anatomies are at least one of (i) magnetic resonance images, or (ii) magnetic resonance images of cancerous breasts.

The method of para. [00232] can be provided, wherein the cancerous anatomies are cancerous breasts.

The method of para. [00232] can be provided, further comprising, prior to the analyzing, performing an imaging procedure on the tissue to obtain the medical image of the tissue.

The method of para. [00232] can be provided, wherein the medical image is a magnetic resonance image of at least one of (i) a human breast, (ii) a human body part that is (a) in need of inspection for possible disease, possible cancer, or possible breast cancer, or (b) afflicted with possible disease, possible cancer, or possible breast cancer.

The method of para. [00232] can be provided, further comprising, prior to the analyzing, performing a histopathology procedure on the tissue to obtain the histopathology sample.

The method of para. [00232] can be provided, wherein the histopathology sample is a digitized histopathology slide of the tissue or a human body part that is (i) in need of inspection for possible disease, possible cancer or possible breast cancer, or (ii) afflicted with possible disease, possible cancer or possible breast cancer

The method of para. [00232] can be provided, wherein the visual feature of the histopathology sample is a visual detail that suggests presence of a cancer.

The method of para. [00232] can be provided, wherein the tissue is a human body part that is (i) in need of inspection for possible disease, possible cancer or possible breast cancer, or (ii) afflicted with breast cancer.

The method of para. [00232] can be provided, wherein the analyzing provides a prediction regarding a medical outcome of the tissue.

The method of para. [00243] can be provided, wherein the prediction regarding the medical outcome of the tissue is a likelihood of (i) a relapse of a disease, cancer or breast cancer, or (ii) a favorable response to a therapy for a condition, cancer or breast cancer present in the tissue.

The method of para. [00243] can be provided, further comprising administering to a subject that provided the sample a therapeutic intervention that corresponds to the prediction regarding the medical outcome of the tissue.

According to another exemplary embodiment of the present disclosure, a method can be provided that comprises:
searching a repository of electronic health records to identify electronic health records that contain at least one input modality obtained from one or more breast diagnostic imaging procedures;
extracting, from the repository, the electronic health records that contain at least one input modality obtained from breast diagnostic imaging procedures that were identified to provide extracted files, wherein each of the extracted health records contains at least one input modality obtained from a breast diagnostic imaging procedure;
standardizing the extracted files to provide standardized, extracted health records;
formatting the standardized, extracted health records in a format that is suitable for training a machine learning procedure, thereby providing formatted, standardized, extracted health records; and
training the machine learning procedure to provide a prediction regarding a medical outcome of a subject based on a breast image of the subject.

The method of para. [00246] can be provided, wherein the searching procedure is performed for more than one repository of the electronic health records.

The method of para. [00246] can be provided, wherein the breast diagnostic imaging procedures comprise at least one of a radiology or a digital pathology.

The method of para. [00246] can be provided, wherein the input modality is imaging data.

The method of para. [00246] can be provided, wherein at least one portion of the extracted health records is associated with more than one input modality.

The method of para. [00246] can be provided, wherein at least one portion of the extracted health records is associated with at least two of MRI, digital pathology, clinical variables, auxiliary clinical variables, or patient outcomes.

The method of para. [00246] can be provided, wherein the prediction regarding the medical outcome of the tissue is a likelihood of (i) a relapse of a disease or breast cancer, (ii) a favorable response to a therapy for a condition, cancer, or breast cancer present in the tissue.

The method of para. [00246] can be provided, wherein the training of the machine learning procedure comprises pretraining at least one of:
(i) the machine learning procedure to perform at least one of a data extraction, a data analysis, or a generation of output,
(ii) a feature extractor of the machine learning procedure to generate a representation of the input modality,
(iii) a feature extractor of the machine learning procedure to generate a multi-modal representation of a plurality of input modalities,
(iv) a feature extractor of the machine learning procedure to generate a multi-modal representation of a plurality of input modalities, and wherein the pretraining is supervised by attaching labels to the input modalities, or
(v) a feature extractor of the machine learning procedure to generate a multi-modal representation of a plurality of input modalities, and wherein the pretraining is self-supervised through a proxy task.

The method of para. [00253] can be provided, wherein the training of the machine learning procedure comprises training the machine learning procedure to map a multi-modal representation to the prediction regarding the medical outcome of the subject based on a multi-modal representation of a plurality of input modalities.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

What is claimed is:

1. A system comprising:
at least one computer processing configuration which utilizes a first feature extractor that is configured to extract at least one image feature from a medical image of a tissue to provide an extracted image feature, and a second feature extractor that is configured to extract at least one histopathology feature from digitized histopathology data of the tissue to provide an extracted histopathology feature,
wherein the at least one computer processing configuration is configured to process:
the at least one extracted image feature and the at least one extracted histopathology feature to provide a multi-modal representation of the tissue, and
the multimodal representation of the tissue with a specific procedure to provide a prediction regarding a medical outcome of the tissue.

2. The system of claim 1, wherein the at least one computer processing configuration is further configured to process the extracted image feature and the at least one extracted histopathology feature with information from an electronic health record corresponding to the tissue to provide the multi-modal representation of the tissue.

3. The system of claim 2, wherein the information from the electronic health record is at least one of (i) a tumor stage of the tissue, or (ii) a status of a molecular biomarker in a subject that provided the tissue.

4. The system of claim 1, wherein the specific procedure is at least one of (i) a machine-learning procedure, (ii) a trained, machine-learning procedure, or (iii) a machine-learning procedure trained on a set of magnetic resonance images of one or more cancerous breasts.

5. The system of claim 1, wherein the medical image is at least one of a magnetic resonance image of a human breast or a human body part that is (i) targeted for an inspection for a possible disease, a cancer or a breast cancer, or (ii) afflicted with a possible disease, a cancer or a breast cancer.

6. The system of claim 1, wherein the extracted image feature is a visual detail that suggests a presence of a cancer.

7. The system of claim 1, wherein the digitized histopathology data of the tissue is an image of a digitized histopathology slide of at least one of (i) the tissue, (ii) a human breast, (iii) a human body part that is targeted for an inspection for a possible disease, a possible cancer or a possible breast cancer, or that is afflicted with a breast cancer.

8. The system of claim 1, wherein the extracted histopathology feature is a visual detail that suggests a presence of a cancer.

9. The system of claim 1, wherein the tissue is a human breast tissue.

10. The system of claim 1, wherein the tissue is a human body part that is targeted for an inspection for at least one of (i) a possible disease, or (ii) a possible breast cancer.

11. The system of claim 1, wherein the tissue is a human breast that is afflicted with a breast cancer.

12. The system of claim 1, wherein the prediction regarding the medical outcome of the tissue is a likelihood of (i) a relapse of a disease or breast cancer, (ii) a favorable response to a therapy for a condition, cancer, or breast cancer present in the tissue.

13. A computer program product comprising a non-transitory, computer-readable medium having a computer-readable program encoded therein, the computer readable program adapted to be executed by a computer processing configuration to implement a procedure comprising:
processing a medical image analysis system, wherein the medical image analysis system comprises:
an image receiving portal;
a first feature extractor;
a histopathology data receiving portal;
a second feature extractor; and
an output function;
receiving by the image receiving portal a medical image of a tissue from an image input source;
extracting by the first feature extractor at least one image feature from the medical image of the tissue to provide an extracted image feature;
receiving by the histopathology data receiving portal digitized histopathology data of the tissue from a histopathology data source;
extracting by the second feature extractor at least one histopathology feature from the digitized histopathology data of the tissue to provide an extracted histopathology feature;
processing the at least one extracted image feature and the at least one extracted histopathology feature to provide a multi-modal representation of the tissue;
processing the multimodal representation of the tissue with a specific procedure to provide a prediction regarding a medical outcome of the tissue; and
outputting by the output function the prediction regarding the medical outcome of the tissue.

14. The computer program product of claim 13, wherein the medical image analysis system further comprises an electronic health record receiving portal; and the method further comprises receiving by the electronic health record receiving portal information from an electronic health record corresponding to the tissue, and
wherein the processing the extracted image feature and the extracted histopathology feature to provide the multi-modal representation of the tissue further comprises processing the information from the electronic health record corresponding to the tissue with the extracted image feature and the at least one extracted histopathology feature to provide a multi-modal representation of the tissue.

15. The computer program product of claim 14, wherein the information from the electronic health record is at least one of (i) a tumor stage of the tissue, or (ii) a status of a molecular biomarker in a subject that provided the tissue.

16. The computer program product of claim 13, wherein the specific procedure is at least one of (i) a machine-learning procedure, (ii) a trained, machine-learning procedure, or (iii) a machine-learning procedure trained on a set of magnetic resonance images of one or more cancerous breasts.

17. The computer program product of claim 13, wherein the medical image is at least one of a magnetic resonance image of a human breast or a human body part that is (i) targeted for an inspection for a possible disease, a cancer or a breast cancer, or (ii) afflicted with a possible disease, a cancer or a breast cancer.

18. The computer program product of claim 13, wherein the extracted image feature is a visual detail that suggests a presence of a cancer.

19. The computer program product of claim 13, wherein the digitized histopathology data of the tissue is an image of a digitized histopathology slide of at least one of (i) the tissue, (ii) a human breast, (iii) a human body part that is targeted for an inspection for a possible disease, a possible cancer or a possible breast cancer, or that is afflicted with a breast cancer.

20. The computer program product of claim 13, wherein the extracted histopathology feature is a visual detail that suggests a presence of a cancer.

21. The computer program product of claim 13, wherein the tissue is a human breast tissue.

22. The computer program product of claim 13, wherein the tissue is a human body part that is targeted for an inspection for at least one of (i) a possible disease, or (ii) a possible breast cancer.

23. The computer program product of claim 13, wherein the tissue is a human breast that is afflicted with a breast cancer.

24. The computer program product of claim 13, wherein the prediction regarding the medical outcome of the tissue is a likelihood of (i) a relapse of a disease or breast cancer, (ii) a favorable response to a therapy for a condition, cancer, or breast cancer present in the tissue.

25. A method comprising:
extracting at least one image feature from a medical image of a tissue to provide an extracted image feature;
extracting at least one histopathology feature from digitized histopathology data of the tissue to provide an extracted histopathology feature;
processing the at least one extracted image feature and the at least one extracted histopathology feature to provide a multi-modal representation of the tissue; and
processing the multimodal representation of the tissue with a specific procedure to provide a prediction regarding a medical outcome of the tissue.
